(19) United States
(12) Patent Application Publication
(10) Pub. No.: US 2017/0327898 A1
(43) Pub. Date: Nov. 16, 2017

(54) OVARIAN CARCINOMA DETECTION AND PROPHYLAXIS

(71) Applicant: THE JOHNS HOPKINS UNIVERSITY, Baltimore, MD (US)

(72) Inventors: Victor Velculescu, Dayton, MD (US); Eniko Papp, Baltimore, MD (US); Vilmos Adleff, Baltimore, MD (US)

(21) Appl. No.: 15/596,517

(22) Filed: May 16, 2017

Related U.S. Application Data
(60) Provisional application No. 62/337,198, filed on May 16, 2016.

(51) Int. Cl. C12Q 1/68; G06F 19/00; G06F 19/18

(52) U.S. Cl. CPC C12Q 1/6886; G06F 19/3481; G06F 19/3431; C12Q 2600/118; C12Q 2600/156; G06F 19/18

(57) ABSTRACT

The evolutionary origin of high-grade serous ovarian carcinoma remains largely unknown. The vast majority of tumor-specific genomic alterations from ovarian cancers are present in fallopian tube STIC lesions (average of 55 sequence alterations per tumor), including those affecting TP53, BRCA1, BRCA2 or PTEN genes. A quantitative evolutionary analysis indicated that tumors of the fallopian tube were the likely precursors of ovarian cancer and could directly give rise to metastatic lesions. These analyses suggest that there may be less than two years between the development of a STIC and the initiation of fallopian tube tumors, ovarian tumors or other metastases. Thus there may be a short window between the development of a STIC and the initiation of ovarian tumors or other metastases, highlighting the importance of the prevention, early detection and therapeutic intervention of this disease.

### REFERENCE TO RELATED APPLICATIONS

This invention application claims the benefit of U.S. Provisional Patent Application No. 62/337,198, filed on May 16, 2016, and is hereby incorporated by reference for all purposes as if fully set forth herein.

This invention was made with government support under CA121113 awarded by National Institutes of Health. The government has certain rights in the invention.

### TECHNICAL FIELD OF THE INVENTION

This invention is related to the area of cancer. In particular, it relates to ovarian cancer and associated gynecological cancers.

### BACKGROUND OF THE INVENTION

Ovarian cancer is the leading cause of death from gynecologic cancers^{1,2}. The 5-year survival is less than 30% and has not improved significantly over the last 30 years³. Despite significant efforts, various screening and therapeutic strategies have generally not led to improved overall survival^{4,5}. One of the major challenges to improved diagnostic and therapeutic intervention in ovarian cancer has been a limited understanding of the natural history of the disease. High grade serous ovarian carcinoma (HGSOC) is the most common histologic subtype of ovarian cancer, accounting for three quarters of ovarian carcinoma⁶⁻⁹. Genomic analyses of HGSOC have identified genetic alterations in TP53, BRCA1/2, PTEN and other genes although few of these discoveries have affected clinical care^{10,11}. HGSOC is diagnosed at advanced stages in approximately 70% of cases, and these women have a significantly worse outcome than those with early stage disease. Until recently, the prevailing view of HGSOC was that it developed from the ovarian surface epithelium.

However, early in situ lesions that arise from the ovarian surface epithelium and progress to invasive HGSOC have never been reproducibly identified.

Insights into the pathogenesis of HGSOC have emerged from investigating the prevalence of occult ovarian and fallopian tube carcinomas in women with germline mutations of BRCA1/2 genes¹²⁻¹⁶. Potential precursor lesions of HGSOC were identified in the fimbriae of the fallopian tubes removed as part of prophylactic surgery¹⁵. Such lesions, including a TP53 mutant single-cell epithelial layer (TP53 signature) and serous tubal in situ carcinoma (STIC)^{16,17}, have been identified in patients with advanced stage sporadic HGSOC of the ovary, fallopian tube and peritoneum¹⁷. Immunohistochemical as well as targeted sequencing analyses have shown that fallopian tube lesions harbor the same TP53 mutation as surrounding invasive carcinomas¹⁶⁻²⁰. These analyses suggest a clonal relationship among such tumors but given the limited number of genes analyzed do not conclusively identify the initiating lesions nor exclude the possibility of fallopian tube metastases from primary ovarian carcinomas^{20,21}.

There is a continuing need in the art for markers and treatments that will permit better detection, treatment, and prophylaxis of gynecological cancers.

### SUMMARY OF THE INVENTION

According to one aspect, a method for reducing risk of ovarian cancer is provided. An option for surgical removal of the fallopian tubes without oophorectomy is offered or recommended to a patient at high risk of ovarian cancer.

According to another aspect, a method is provided for reducing risk of ovarian cancer. An option for surgical removal of the fallopian tubes without oophorectomy is offered or recommended to a patient who is a candidate for obtaining a tubal ligation as a contraceptive measure.

According to another aspect, a method is provided for reducing risk of omental cancer or metastasis. An option for surgical removal of the fallopian tubes without oophorectomy is offered or recommended to a patient who is a candidate for obtaining a hysterectomy for a benign cause.

According to another aspect, a method is provided for detecting an increased risk of ovarian cancer and metastases. An examination is conducted of at least 3 sections of a pair of removed fallopian tubes that were removed for a benign condition, for a risk-reducing bilateral salpingectomy, or for a gynecological cancer.

According to another aspect, a method is provided of characterizing a lesion in fallopian tubes or ovaries of a patient. Loss of heterozygosity of a marker selected from the group consisting of p53, PTEN, BRCA1, and BRCA2 is tested for and detected in a sample of the lesion.

According to another aspect, a method is provided of characterizing a lesion in fallopian tube of a patient. A mutation in a gene selected from the group consisting of: CWC22, DUSP27, KIF13A, PIK3R5, TTN, WDFY4, and WDR11 is tested for and detected.

According to another aspect, a method is provided of detecting or characterizing a lesion in fallopian tube of a patient. A mutation in a gene selected from the group consisting of: CWC22, DUSP27, KIF13A, PIK3R5, TTN, WDFY4, and WDR11 is tested for and detected in a PAP smear or liquid PAP smear sample.

These and other aspects and embodiments which will be apparent to those of skill in the art upon reading the specification provide the art with

### BRIEF DESCRIPTION OF THE DRAWINGS

FIG. 1 Schematic of sample isolation and next-generation sequencing analyses. (Top panel) Tumor sites analyzed from Patient 1 with stage III HGSOC. For each sample, slides were stained with hematoxylin and eosin as well as analyzed by immunohistochemical staining of TP53. (Middle panel) Tumor samples were microdissected for genomic analyses. For microdissection for STIC and TP53 signature lesions, tumor cells were identified using immunohistochemical staining of TP53 and isolated through laser capture microdissection. (Bottom panel, left) Next-generation sequencing analyses were performed for tumor specimens using either whole-exome or targeted analyses focused on 120 genes. (Bottom panel, right) Somatic mutations and chromosomal alterations were used to evaluate tumor evolution using the tumor subclonality phylogenetic reconstruction algorithm SCHISM, allelic imbalance clustering algorithms, and to determine a timeline for tumor progression.

FIG. 2A-2B. Somatic mutation profiles among different tumor lesions. Somatic mutations detected by whole-exome analyses are indicated as colored cells in rows for Patient 1 (FIG. 2A) and Patient 2 (FIG. 2B). The tumor samples analyzed for each patient are indicated in columns (TP53 sig, TP53 signature; STIC, serous tubal intraepithelial carcinoma). For ovarian tumors in FIG. 2A and STIC lesions in FIG. 2B multiple blocks are indicated, including one ovarian tumor where multiple sections were analyzed after hematoxylin and eosin staining or after immunohistochemistry (IHC) staining of TP53. These analyses indicated that staining methods did not affect detection of somatic alterations. The color of mutations indicates the degree of relatedness among tumor samples: red, shared among all tumor samples with TP53 highlighted at the top row; green, shared among all tumor samples except TP53 signature lesion; purple, shared among left fallopian tube tumor and omental metastasis; blue indicates mutations that were first detected in the ovarian tumors; and gray indicates mutations that were only detected in omental metastatic lesions.

FIG. 3A-3B. Genome-wide allelic imbalance analysis. Allelic imbalance across the genome of each tumor sample (Left Panel) is indicated for Patient 1 (FIG. 3A) and Patient 2 (FIG. 3B). The genome was divided into chromosome bands and for each band the minor allele (B-allele) frequency values were compared between tumor and normal samples using the 15,000 whole-exome germline heterozygous SNPs. Purple bands indicate region of imbalance defined as chromosome bands with a p-value less than or equal to 0.05, and a minimum mean difference of 0.1 between b-allele frequencies of tumor and normal samples. Chromosome bands with less than 5 informative SNPs were considered to be of unknown status as indicated in gray. White boxes indicate normal copy number. Regions encompassing PTEN, BRCA1/2 and TP53 are highlighted. To investigate the evolutionary relationship among tumor samples, genomic positions involved in allelic imbalance were used to generate cluster dendrograms (Right Panel). Tumors with the highest degree of similarity are grouped in short-branched clusters and further connected to similar cases generating higher-order clusters of decreasing similarity as indicated by longer branches.

FIG. 4A-4B. Schematic of tumor evolution. Tumor subclonal hierarchies (Right panels) are indicated in in relation to anatomic sites (Left panels) for Patient 1 (FIG. 4A) and Patient 2 (FIG. 4B). Compartments of mutations containing a sublcone are illustrated by a tree node (blue circle) and mutations acquired by the cells in the progeny nodes that distinguish them from the cells in the parental node are represented by an edge (arrow). The optimal hierarchy among subclones is determined by evaluating all possible relationships and identifying the simplest model that can explain the observed nodes. A). The optimal hierarchy (left panel) for Patient 1 and 2 are illustrated by indicated tree nodes (Right panels). Gene mutations corresponding to each edge are described in Supp. Table S4.

FIG. 5 Loss of heterozygosity analyses for Patient 1, chromosomes 1-11. The graphs represent B allele frequencies (BAFs) for the indicated chromosomes. A value of 0.5 indicates a heterozygous genotype (AB) whereas allelic imbalances in tumor samples are observed as a deviation from 0.5. BAF values of 0 typically indicate loss of heterozygosity, although normal contaminating tissue may limit the minimum observed value. Graphs for Patient 1 include left fallopian tube STIC, left fallopian tube tumor, left ovarian tumor block A4, left ovarian tumor block A7, right ovarian tumor, rectal metastasis, appendiceal metastasis, and omental metastasis.

FIG. 6 Loss of heterozygosity analyses for Patient 1, chromosomes 12-X. The graphs represent B allele frequencies (BAFs) for the indicated chromosomes. A value of 0.5 indicates a heterozygous genotype (AB) whereas allelic imbalances in tumor samples are observed as a deviation from 0.5. BAF values of 0 typically indicate loss of heterozygosity, although normal contaminating tissue may limit the minimum observed value. Graphs for Patient 1 include left fallopian tube STIC, left fallopian tube tumor, left ovarian tumor block A4, left ovarian tumor block A7, right ovarian tumor, rectal metastasis, appendiceal metastasis, and omental metastasis.

FIG. 7 Loss of heterozygosity analyses for Patient 2, chromosomes 1-11. The graphs represent B allele frequencies (BAFs) for the indicated chromosomes. A value of 0.5 indicates a heterozygous genotype (AB) whereas allelic imbalances in tumor samples are observed as a deviation from 0.5. BAF values of 0 typically indicate loss of heterozygosity, although normal contaminating tissue may limit the minimum observed value. Graphs for Patient 2 include STIC block D1, STIC block D2, STIC block D3, right fallopian tumor, right ovarian tumor, sigmoid metastasis, rectal metastasis.

FIG. 8 Loss of heterozygosity analyses for Patient 2, chromosomes 12-X. The graphs represent B allele frequencies (BAFs) for the indicated chromosomes. A value of 0.5 indicates a heterozygous genotype (AB) whereas allelic imbalances in tumor samples are observed as a deviation from 0.5. BAF values of 0 typically indicate loss of heterozygosity, although normal contaminating tissue may limit the minimum observed value. Graphs for Patient 2 include STIC block D1, STIC block D2, STIC block D3, right fallopian tumor, right ovarian tumor, sigmoid metastasis, rectal metastasis.

FIG. 9 Loss of heterozygosity analyses for Patient 3. The graphs represent B allele frequencies (BAFs) for the indicated chromosomes for the STIC lesion. A value of 0.5 indicates a heterozygous genotype (AB) whereas allelic imbalances in tumor samples are observed as a deviation from 0.5. BAF values of 0 typically indicate loss of heterozygosity, although normal contaminating tissue may limit the minimum observed value.

FIG. 10 Loss of heterozygosity analyses for Patient 4. The graphs represent B allele frequencies (BAFs) for the indicated chromosomes for the STIC lesion. A value of 0.5 indicates a heterozygous genotype (AB) whereas allelic imbalances in tumor samples are observed as a deviation from 0.5. BAF values of 0 typically indicate loss of heterozygosity, although normal contaminating tissue may limit the minimum observed value.

FIG. 11 (Supplementary Table 1) Summary of Samples and Next-Generation Sequencing Analyses.

FIG. 12 (Supplementary Table 2) Targeted Sequencing Panel.

FIG. 13 (Supplementary Table 3) Somatic Sequence Alterations.

FIG. 14 (Supplementary Table 4) Somatic Sequence Alterations used for Evolutionary Cluster Analyses.

FIG. 15 (Supplementary Table 5) Recurrent Somatic Sequence Alterations.

### DETAILED DESCRIPTION OF THE INVENTION

The inventors utilized genome-wide sequence and structural analyses of multiple ovarian tumors from the same individual to examine the origins of HGSOC. They had previously shown that the acquisition of somatic alterations can be used as a molecular marker in the development of human cancer²². Here, they examine whether the compendium of somatic alterations identified in different lesions may provide insights into the evolutionary relationship between primary ovarian carcinomas, fallopian tube lesions, and ovarian cancer metastases.

A patient at high risk of ovarian cancer is one who has one or more genetic or physiological conditions including but not limited to BRCA1 or BRCA2 mutations, advanced age, obesity, reproductive history, fertility drug use, androgen use, estrogen use, family cancer syndrome, HNPCC mutation in MLH1, MLH3, MSH2, MSH6, TGFBR2, PMS1, or PMS2, Putz-Jeghers syndrome, MUTYH-associated polyposis, and personal history of breast cancer. A patient may have 2, 3, 4, 5, or more of such idicators.

Offering or recommending to a patient may be done in writing, electronically, and/or orally. The delivery of the offer or recommendation may be done by a genetic counselor, a primary care physician, a clinical laboratory, a nurse, a surgeon, etc.

Mutations and loss of heterozygosity can be tested and detected using any means known in the art. Specific mutations and genome segments may be targeted and assayed. Alternatively, the testing may be non-targeted, such as whole genome sequencing or whole exome sequencing. Probes and primers that are specific for a particular gene or particular mutation may be used. In one aspect, the probes and/or primers may be attached to a solid support, such as an array or a bead. In another aspect, the probes and/or primers may incorporate modified, non-naturally occurring nucleotides such as phosphorodiamidate morpholino nucleotides to diminish degradation.

Pap smear and liquid Pap smear samples can be used as test samples. Collecting such samples is known in the art.

Given the unique nature of the multiple samples we examined from each patient, our study may have certain limitations not typical of genome-wide efforts. First, the small size of the tumor samples compared to surrounding non-neoplastic tissue could potentially lead to low mutation cellularity. The high mutant allele fraction of TP53 in samples of Patients 1, 2 and 4 (average of 54-85%) indicates that this issue was largely overcome through laser capture microdissection. Second, the small number of cells in TP53 signature samples may have limited our genomic analyses for these lesions. Although these samples were not used in the SCHISM or allelic imbalance analyses, the observation that all sequence changes in TP53 signatures were also present in STIC and other tumors is consistent with our evolutionary model and suggests that these cells are likely to represent a parental clone of other neoplastic lesions. Third, our analysis was limited to ovarian tumors where STICs and other concomitant lesions were identified, and may therefore not be representative of all HGOCs. Although STIC lesions are not identified in ˜40% of sporadic HGOCs, this absence may reflect an incomplete sampling of the fallopian tube or the overgrowth of the STIC lesion by the carcinoma²⁷. Fourth, as in any evolutionary analyses, the genomic alterations we observed provide the most likely model of tumor development but do not exclude the possibility of other relationships. Nevertheless, the comprehensive analyses of somatic alterations suggest that models where the ovarian cancer or metastatic lesions seed the fallopian tube tumors^{19,20} are unlikely to be consistent with the observed alterations.

Despite these potential limitations, the data we have obtained provide important insights into the etiology of ovarian cancer and have significant implications for the prevention, early detection and therapeutic intervention of this disease. The results suggest that ovarian cancer is a disease of the fallopian tubes, with the development of STICs and fallopian tube tumors as early events. Our observations support the notion that formation of a cancer in the ovaries represents a seeding event from a primary tumor in the fallopian tube that already contains key driver alterations, including those in TP53, PI3K pathway, and BRCA1/2 genes. In some cases metastatic lesions may also be seeded directly from the fallopian tube lesion, completely bypassing the ovaries (e.g. the omental metastasis of Patient 1). In this manner, the ovarian cancer and other metastases may be equivalent sites of seeding from a fallopian tube tumor. These observations can help explain why most HGSOC patients are diagnosed at advanced stage (III/IV) with pelvic and peritoneal spread of disease, and why among asymptomatic BRCA germline mutation carriers half of the cases diagnosed with adnexal neoplasia have already seeded to pelvis or peritoneum (>IA)²⁸.

Our genomic analyses are consistent with population-based studies of the effects of salpingectomy on the risk of ovarian cancer. Prophylactic bilateral salpingo-oophorectomy has been shown to reduce the risk of developing ovarian cancer in BRCA mutation carriers to below 5%^{29,30}. Likewise, bilateral salpingectomy, performed as a contraceptive method instead of tubal sterilization, reduced the risk of ovarian cancer by 61% at 10 years³¹. Our study provides a mechanistic basis for these observations and has specific implications for clinical management in prevention of ovarian cancer. These include the following: 1) for women who are not considered to be at high risk but who undergo surgery for benign uterine causes, total abdominal hysterectomy and bilateral salpingectomy with sparing of the ovaries should be considered³², 2) bilateral salpingectomy may be a preferred contraceptive alternative to tubal ligation, and 3) for high-risk women, bilateral salpingectomy with delayed oophorectomy should be considered³³. The dual concepts in these recommendations are that removal of the fallopian tubes (rather than the ovaries) eliminates the underlying cellular precursors of ovarian cancer, and that preservation of the ovaries provides long term benefits due to decreased risk and fatalities from coronary heart disease and other illnesses³⁴.

Our observations also have implications for improved detection of ovarian cancer. Unfortunately, less than 1.25% of HGSOC are confined to the ovary at diagnosis²¹. Earlier detection of this disease is likely to benefit from the identification of a precursor lesion, as has been the case for many other tumor types. Our data suggest that fallopian tube neoplasia is not only the origin of ovarian serous carcinogenesis, but can directly lead to cancer of the ovaries and of other sites. Currently, the typical histopathologic evaluation of fallopian tubes typically involves a cursory evaluation of one or two representative sections. Our study suggests that systematic sectioning and extensive examination of total fallopian tubes¹⁵ should become common practice in pathology, and not confined to academic tertiary care centers. Depending on whether the fallopian tubes are removed for benign conditions, risk-reducing bilateral salpingectomy, or gynecological cancers, specific examination protocols should be applied^{15,35}. Given the window of time that appears to exist between the formation of fallopian tube lesions and development of ovarian cancer, these insights open the prospect of novel approaches for screening. Such approaches may be especially important given the limited therapeutic options currently available for ovarian cancer^{4,5}. Recent advances for sensitive detection of genetic alterations in blood-based liquid biopsies, pap smears, and other bodily fluids^{36,37} may provide opportunities in early diagnosis and intervention.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Methods

### Specimens Obtained for Sequencing Analysis

The study was approved by the Institutional Review Board at Brigham and Women's Hospital and all patients gave informed consent before inclusion. Two patients with stage III HGSOC, in whom a serous tubal in situ carcinoma (STIC) was identified in their fallopian tubes (FT), were included. In addition, two patients with BRCA deleterious mutation that underwent prophylactic bilateral salpingoophorectomy and in whom a STIC was identified in their FT were included. Formalin-fixed paraffin embedded (FFPE) blocks were retrieved from the pathology files at Brigham and Women's Hospital within the 3 months following surgical diagnosis and stored at 4° C. to slow down nucleic acids degradation. All the cases were reviewed by a gynecologic pathologist (MH and DL) that confirmed the diagnosis of STIC and/or TP53 signature in the FT. Slides from each FFPE block to microdissected, including early lesions, invasive tumors and metastases, were stained with Hematoxylin & Eosin (H&E) and p53. In each FT, at least one STIC and/or TP53 signature was identified and microdissected separately. Importantly, STICs were not pooled together even if they were in the same section. They were considered separate STICs.

### Immunohistochemistry p53 Staining for Laser Capture Microdissection

For accurate microdissection of early lesions including STIC and TP53 signature, immunohistochemistry staining of TP53 was specifically adapted for Laser Capture Microdissection (LCM) as previously described³⁸. PEN membrane frame slides Arcturus (Life technologies, Carlsbad, Calif.) were used. Each slide was coated with 350 mL of undiluted poly-L-lysine 0.1% w/v (Sigma, St. Louis, Mo.). For drying, the slides were placed in a slide holder for 60 minutes at room temperature. Tissue sections were cut and mounted on the pretreated membrane slides. Deparaffinization was performed in fresh xylene for 5 minutes twice, followed by 100% ethanol for 2 minutes, 95% for ethanol 2 minutes and 70% ethanol for 2 minutes. Subsequently, the slides were transferred into distilled water for 5 minutes. Heat-epitope antigen retrieval (AR) was performed in Citrate Buffer (Dako, Carpinteria, Calif.) at low temperature (60° C.) for 44 hours instead of 120° C. for 10 minutes to reduce tissue and DNA damage by high temperature. Retrieval solution was pre-warmed to 60° C. before usage. After incubation in the oven, the AR solution was left to cool down to room temperature and the slides were rinsed for 30 seconds in fresh 1X PBS then incubated for 40 minutes with primary antibody anti-p53 (Epitomics, Burlingame) at 1:100 in a humidifying chamber. Before adding the secondary antibody, slides were washed twice for 1 minute in fresh 1X PBS. The secondary antibody, labeled polymer-HRP anti-mouse (Dako EnVision System-HRP (DAB), Carpinteria, Calif.) was applied for 30 minutes. Then, slides were washed twice for 1 minute in fresh 1X PBS. Chromogenic labeling was performed with 3,3-DAB substrate buffer and DAB chromogen (Dako EnVision System-HRP (DAB), Carpinteria, CA) for 5 minutes. Slides were washed again for 30 seconds in fresh distilled water. Dehydration was performed as follows: 70% ethanol for 30 seconds, 95% ethanol for 30 seconds, 100% ethanol for 30 seconds, and xylene for 30 seconds. The stained slides were microdissected within 2 hours with the Arcturus XT LCM system (Life technologies, Carlsbad, Calif.).

### Hematoxylin Staining for LCM

Invasive carcinomas from primary tumors and metastases were microdissected after Hematoxylin staining. Briefly, deparaffinization was performed in fresh xylene for 1 minute twice followed by 100% ethanol for 1 minute, 95% for ethanol 1 minute and 70% ethanol for 1 minute. The slides were transferred into distilled water for 2 minutes before staining with Hematoxylin for 2 minutes. Subsequently, slides were rinsed in distilled water until they became clear before undergoing dehydration in 70% ethanol for 1 minute, 95% ethanol for 1 minute, 100% ethanol for 1 minute and xylene for 1 minute. The stained slides were microdissected within 2 hours.

### Sample Preparation and Next-Generation Sequencing

DNA was extracted from patient whole blood using a QIAamp DNA Blood Mini QIAcube Kit (Qiagen Valencia, Calif.). Genomic DNA from FFPE blocks was extracted from the microdissected tissues using the QlAamp DNA FFPE Tissue kit (Qiagen, Valencia, Calif.). In brief, the samples were incubated in proteinase K for 16 hours before DNA extraction. The digested mixture was transferred to a microtube for DNA fragmentation using the truXTRAC™ FFPE DNA Kit with 10 min shearing time as per the manufacturer's instructions (Covaris, Woburn, Mass.). Following fragmentation, the sample was further digested for 24 hours followed by one hour incubation at 80° C. DNA purification was performed using the QIAamp DNA FFPE Tissue kit following the manufacturer's instructions (Qiagen, Valencia, Calif.). Fragmented genomic DNA from tumor and normal samples were used for Illumina TruSeq library construction (Illumina, San Diego, Calif.) according to the manufacturer's instructions or as previously described³⁹. Exonic or targeted regions were captured in solution using the Agilent SureSelect v.4 kit or a custom targeted panel according to the manufacturer's instructions (Agilent, Santa Clara, Calif.). Paired-end sequencing, resulting in 100 bases from each end of the fragments for exome libraries and 150 bases from each end of the fragment for targeted libraries, was performed using Illumina HiSeq 2000/2500 and Illumina MiSeq instrumentation (Illumina, San Diego, Calif.).

We used gentle shearing in protective medium of 5% SDS for 10 min at 20° C. with AFA (Adaptive Focused Acoustics) sonication on a Covaris instrument performed in a Snap-Cap microTUBE with AFA fiber at 75W peak incidence power (PIP), 20% duty cycle and 200 cycle/burst. The sheared DNA amount (based on recovered amount was from 30-100 sheared DNA ng) or down to 4 mm² 10 μm thick FFPE tissue or FFPE microdissected material.

We combined the following steps in FFPE DNA extraction and sonication: (a) tissue collection from slide with the SDS containing buffer or ATL—after deparaffinization; (b) Prot K digestion in the same buffer (c) Shearing on the Covaris instrument still in the same buffer; (d) Extraction with the Qiagen kit from the same buffer and producing a purified sheared DNA library ready for NGS library preparation. By combining these steps the losses of DNA are minimized and the method is amenable to prep very low amount of sheared DNA.

### Primary Processing of Next-Generation Sequencing Data and Identification of Putative Aomatic Mutations

Somatic mutations were identified using VariantDx⁴⁰ custom software for identifying mutations in matched tumor and normal samples. Prior to mutation calling, primary processing of sequence data for both tumor and normal samples were performed using Illumina CASAVA software (v1.8), including masking of adapter sequences. Sequence reads were aligned against the human reference genome (version hg18) using ELAND. Candidate somatic mutations, consisting of point mutations, insertions, and deletions were then identified using VariantDx across the either the whole exome or regions of interest³⁹. For samples analyzed using targeted sequencing, we identified candidate mutations that were altered in >10% of distinct reads. For samples analyzed using whole exome sequencing, we identified candidate mutations that were altered in >10% of distinct reads with ≧5 altered reads in at least one sample and where the ratio of the coverage of the mutated base to the overall sequence coverage of that sample was >20%. Identified mutations were reported as present in other samples of the same patient if the mutation was present in at least 2 distinct altered reads. Mutations present in polyN tract ≧5 bases, or those with an average distinct coverage below 50× were removed from the analysis.

An analysis of each candidate mutated region was performed using BLAT. For each mutation, 101 bases including 50 bases 5′ and 3′ flanking the mutated base was used as query sequence (genome.ucsc.edu/cgi-bin/hgBlat). Candidate mutations were removed from further analysis, if the analyzed region resulted in >1 BLAT hits with 90% identity over 70 SCORE sequence length. All candidate alterations were examined by visual inspection, and any alteration present.

### Genome-Wide Allelic Imbalance Analysis

An analysis of allelic imbalance across the genome of each tumor sample was performed to identify genomic regions with potential copy number aberrations. The reference genome (hg18 assembly) was divided into 317 intervals defined based on chromosome cytobands as observed on Giemsa-stained chromosomes (UCSC table browser, most recent cytoband table last updated on 2009-06-12), using double digit loci level of resolution (e.g. 3p12 for sub-band 2 on band 1 on the short arm of chromosome 3). Each chromosome harbored 26 to 6 chromosome bands. A set of genomic positions with germline heterozygous SNPs were identified using the normal tissue sample in each patient (15.5 k positions in patient 1, and 15.4 k in patient 2). Occurrences of the reference and alternate allele in the read pileup at each position in a tumor sample were recorded. These values defined the total coverage and the b-allele fraction of each SNP in each sequenced tumor sample.

In a given tumor sample, the status of each chromosome band with regards to allelic imbalance was determined as follows using custom R scripts (R statistical computing environment, version 3.1.2). Germline positions were filtered to keep those with a minimum coverage of 10 reads in both normal and tumor sample. Chromosome bands with less than 5 informative positions were considered to be of unknown status. In the remaining bands, a one-tailed paired t-test compared the b-allele frequency of positions between the tumor and normal sample from the same patient. Furthermore, the mean difference between the two groups was recorded. The resulting p-values for chromosome bands in each tumor sample were corrected for multiple hypothesis testing using Bonferroni method. Chromosome bands with an adjusted p-value less than or equal to 0.05, and a minimum mean difference of 0.1 between b-allele frequencies of tumor and normal samples were declared as regions of allelic imbalance.

To define genome-wide similarity of tumor samples in terms of their copy number profile, each tumor sample was coded as a binary vector, corresponding to its allelic imbalance status in chromosome bands across the genome. The euclidean distance between the above imbalance vectors from each pair of tumor samples was calculated and used as a distance measure in hierarchical agglomerative clustering in R.

### Subclonal Hierarchy Analysis

The tumor subclonality phylogenetic reconstruction algorithm SCHISM 1.0.0²³ was used to infer tumor subclonal hierarchies from the set of confidently called somatic mutations in each patient. Mutation cellularity was estimated from observed read counts and was narrowed down to exclude those in regions of allelic imbalance in any sample. The local allelic imbalance status in the interval around each mutation was identified by using the 10 closest germline heterozygous SNPs and a procedure identical to chromosome band analysis described above. Next, each mutation was called as present or absent in each of the 10 samples from patient 1 and 6 samples from patient 2. Mutations were clustered by a greedy algorithm based on their joint presence and absence across all samples in each patient. The tumor content (i.e. purity) in each tumor sample was estimated as the read count fraction of TP53 mutation in each patient. Both patients harbor a single TP53 mutation that was present in all tumor samples; we assume it is diploid with wild-type allele lost as observed by the LOH of chromosome 17. SCHISM was run with the above inputs and default parameter settings to infer the order of mutation clusters and thus define subclonal hierarchy in each patient. SCHISM software is freely available for non-commercial use at karchinlab.org/appSchism.

Estimating an Evolutionary Timeline

Following the approach of Jones et al.²⁶, the observed data are the number of somatic mutations in the STIC (N1), the number of mutations in the metastasis (N2), and the age at which the patient was diagnosed (T2). Unknown is the birth date (T1) of the cell that was the last common ancestor of the STIC and the metastasis. Assuming the mutation rate of somatic passenger mutations and the length of the cell cycle is constant, the number of somatic mutations in the metastasis cell that were present in the STIC follows a binomial distribution with parameters N2 and probability T1/T2. As Ti is unknown, we posit a conjugate beta probability distribution on the rate T1/T2 with shape parameters (a) and (b) estimated from previous studies as described below. The posterior distribution of T1/T2 is beta (a+N1, b+N2−N1) from which 90% highest posterior density intervals can be constructed with point estimates for the birthdate reported as the modal ordinate. For simplicity, we refer to the highest posterior density as a confidence interval. To construct a prior for 71112, we draw on a previous study of four colorectal cancer patients²⁶ where a small number of additional passenger mutations were acquired by the cell that gave birth to the metastasis, On average, 95% of the mutations in the original adenocarcinoma were present in the metastases. We center the mean for the beta prior at 0.95 using shape parameters a=34 and b=1.6. Our prior is equivalent to one patient having 34 passenger somatic mutations in the original lesion and 1,6 additional mutations to be acquired by cells that gave birth to the metastases. In addition to the beta-binomial model, we also modeled the evolutionary timeline for the accumulation of passenger somatic mutations as a Poisson process as described in Yachida et al.²² This alternative model provided qualitatively similar estimates for the time between STIC formation and development of metastases.

### EXAMPLE 2

### Overall Approach

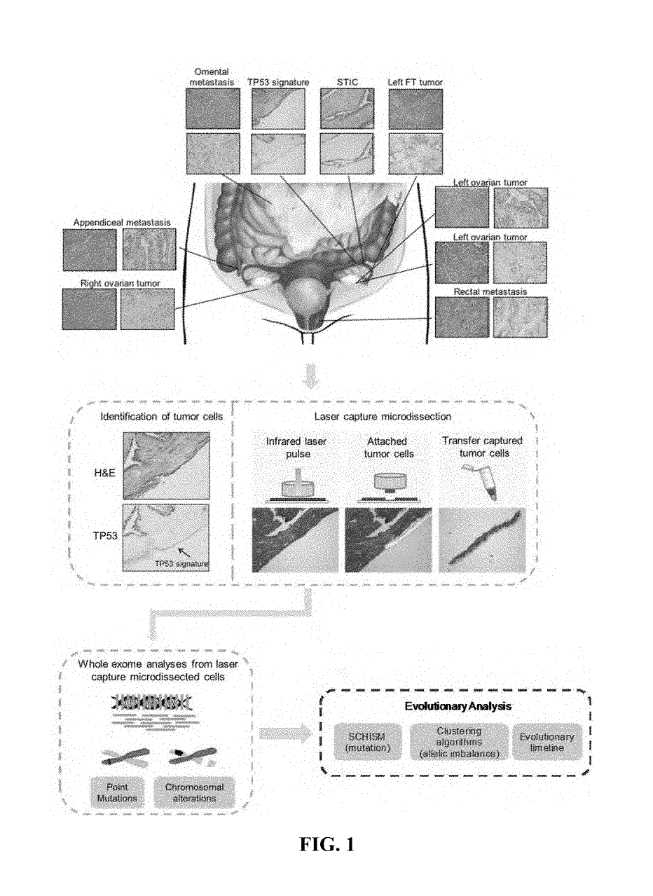

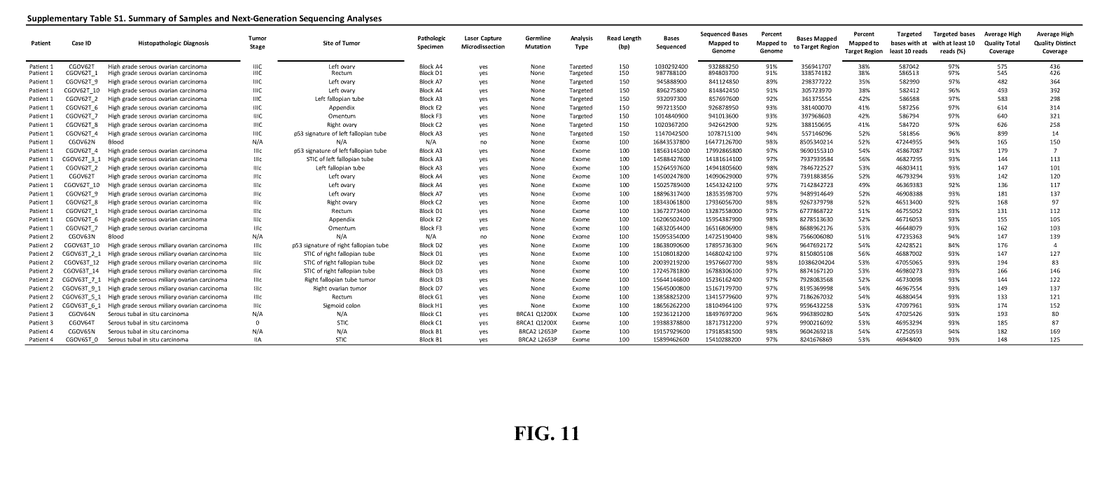

To elucidate the relationship among tumors in patients with HGSOC, we performed whole-exome sequencing of 28 samples from four patients with multiple ovarian cancer lesions (FIG. 11, Supplementary Table S1). We included two patients with stage IIIC HGSOC in whom STIC lesions were identified (FIG. 11). For Patient 1, we analyzed the ovarian tumor of the left ovary and lesions of the left fallopian tube, including a TP53 signature, one STIC lesion, and fallopian tube tumor (FIG. 1, FIG. 11). We also evaluated from this patient a tumor of the right ovary as well as rectal, appendiceal and omental metastases. For Patient 2, we analyzed the tumor of the right ovary, lesions from the right fallopian tube including a TP53 signature, three different STIC lesions, a fallopian tube tumor, and lesions of the rectum and sigmoid colon (FIG. 11). In addition, we included two patients (Patients 3 and 4) with germline pathogenic BRCA mutations that underwent prophylactic bilateral salpingo-oophorectomy and in whom STIC lesions were identified in their fallopian tubes. For all patients, laser capture microdissection was used to isolate lesions after immunohistochemistry staining of TP53 in STIC and TP53 signatures or after hematoxylin staining of other samples. Whole blood or normal fallopian tube epithelium were used as control samples.

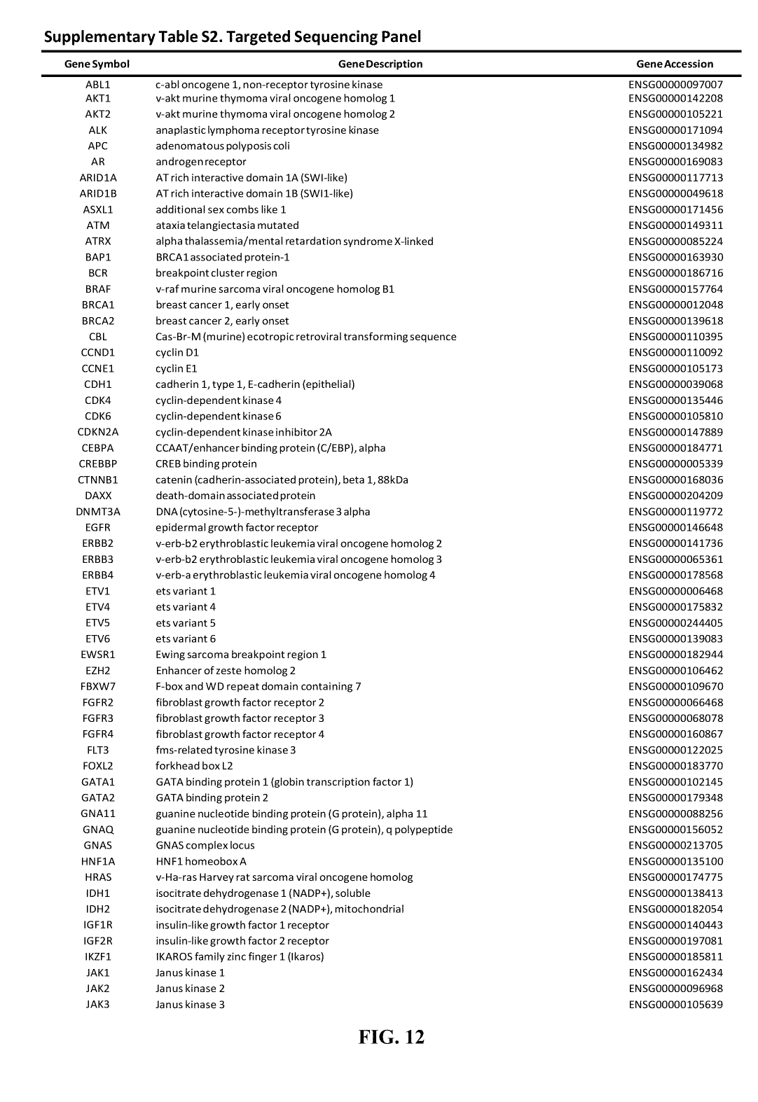

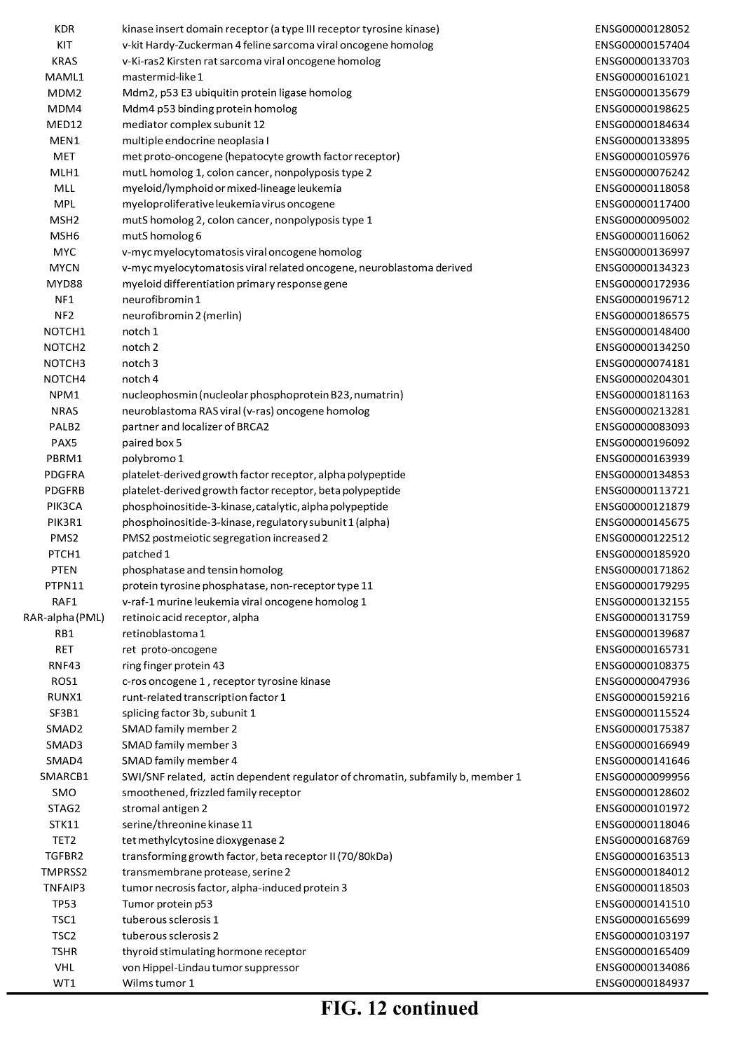

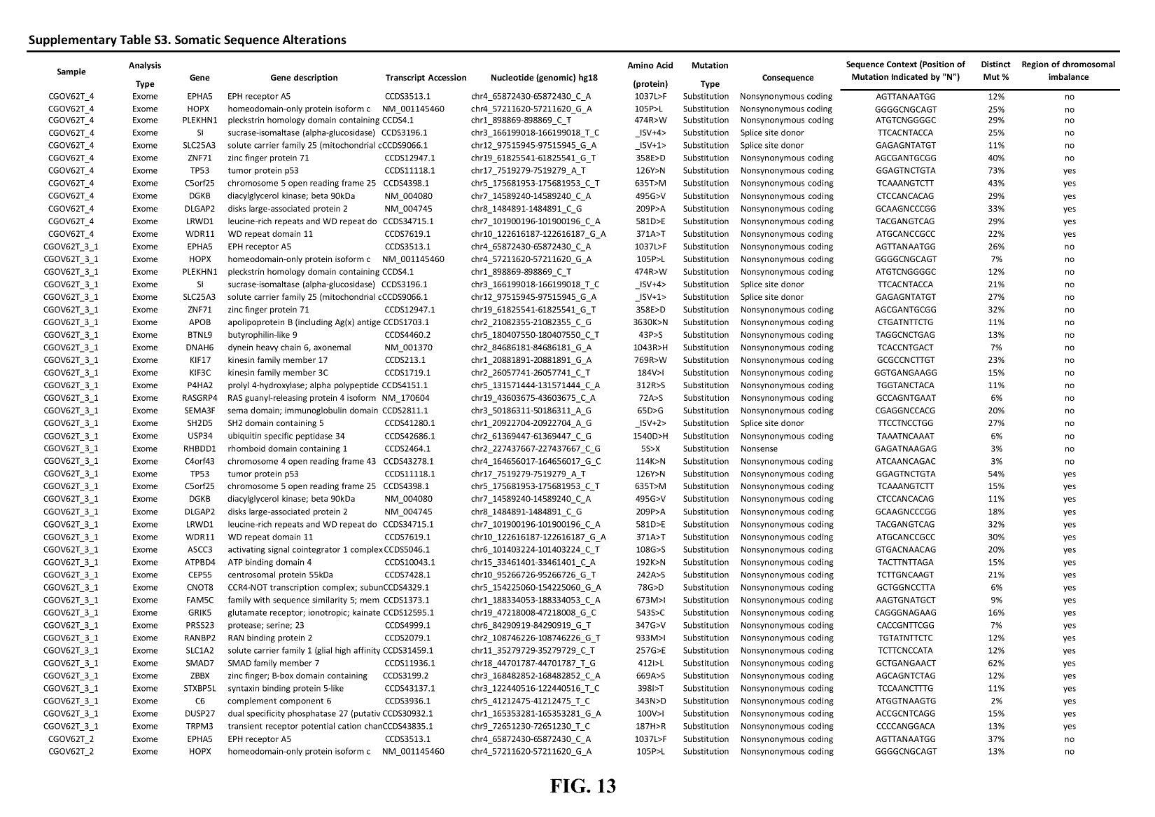

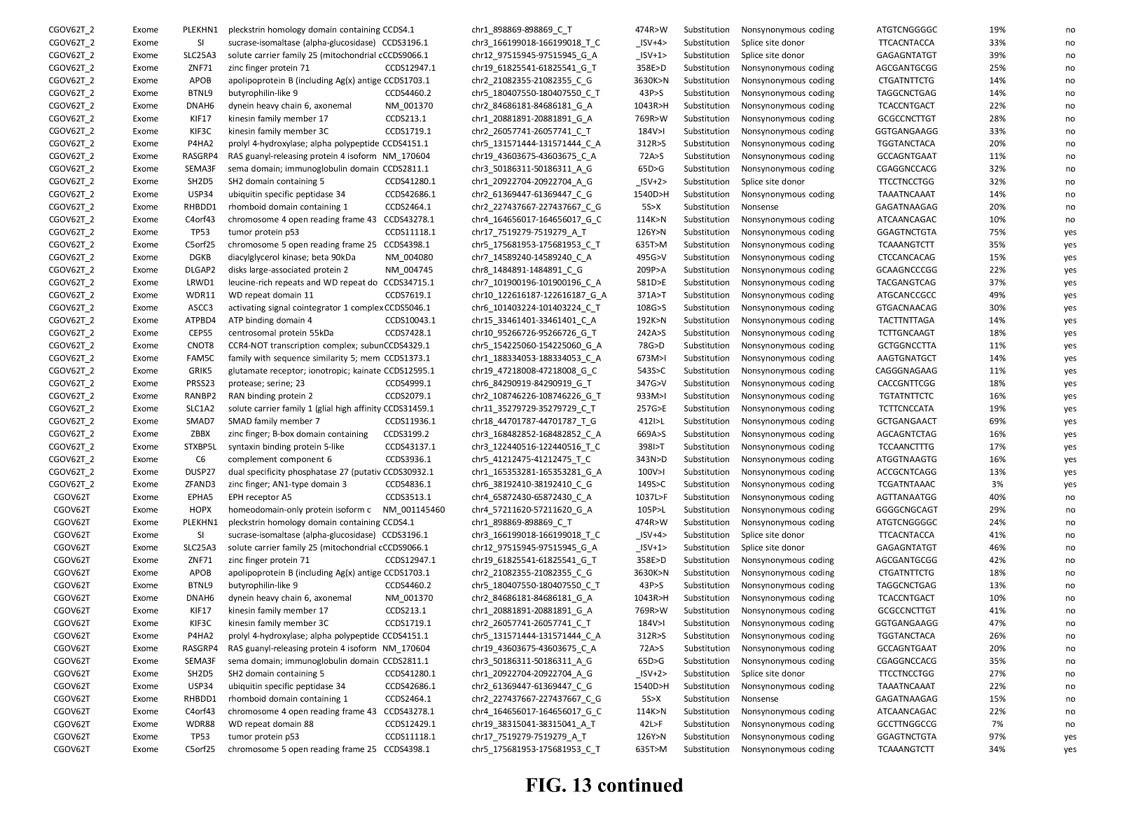

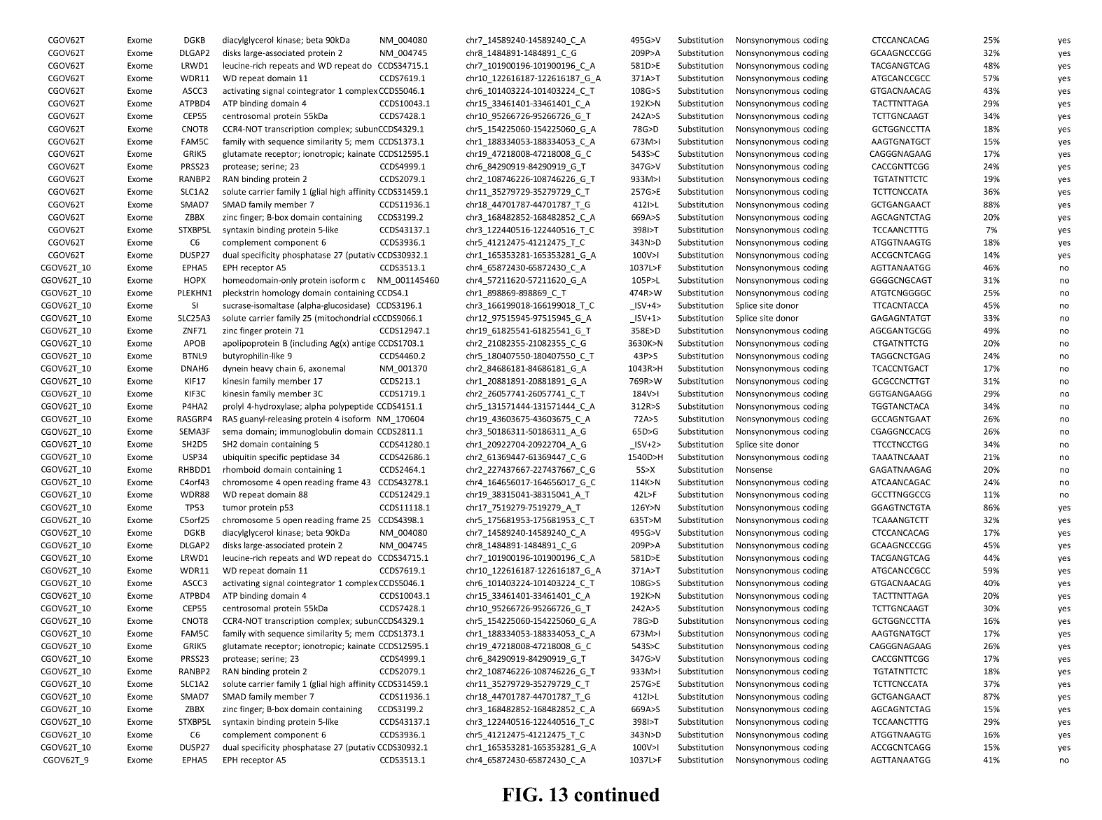

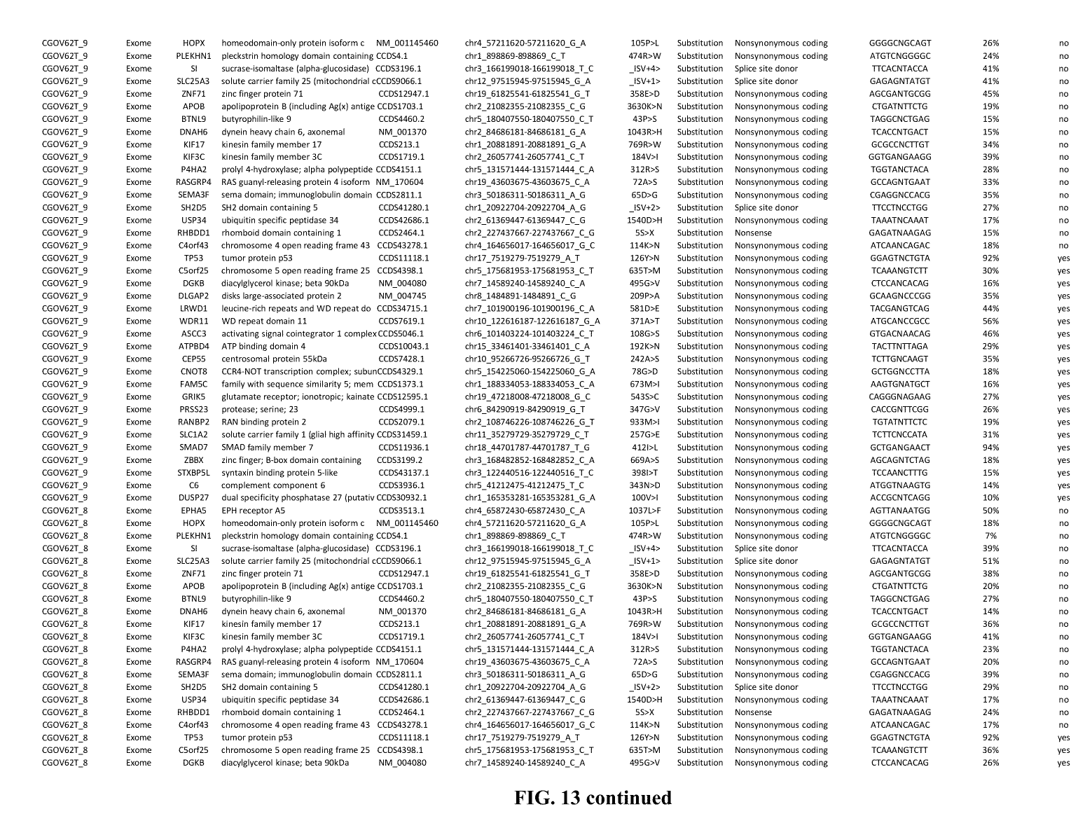

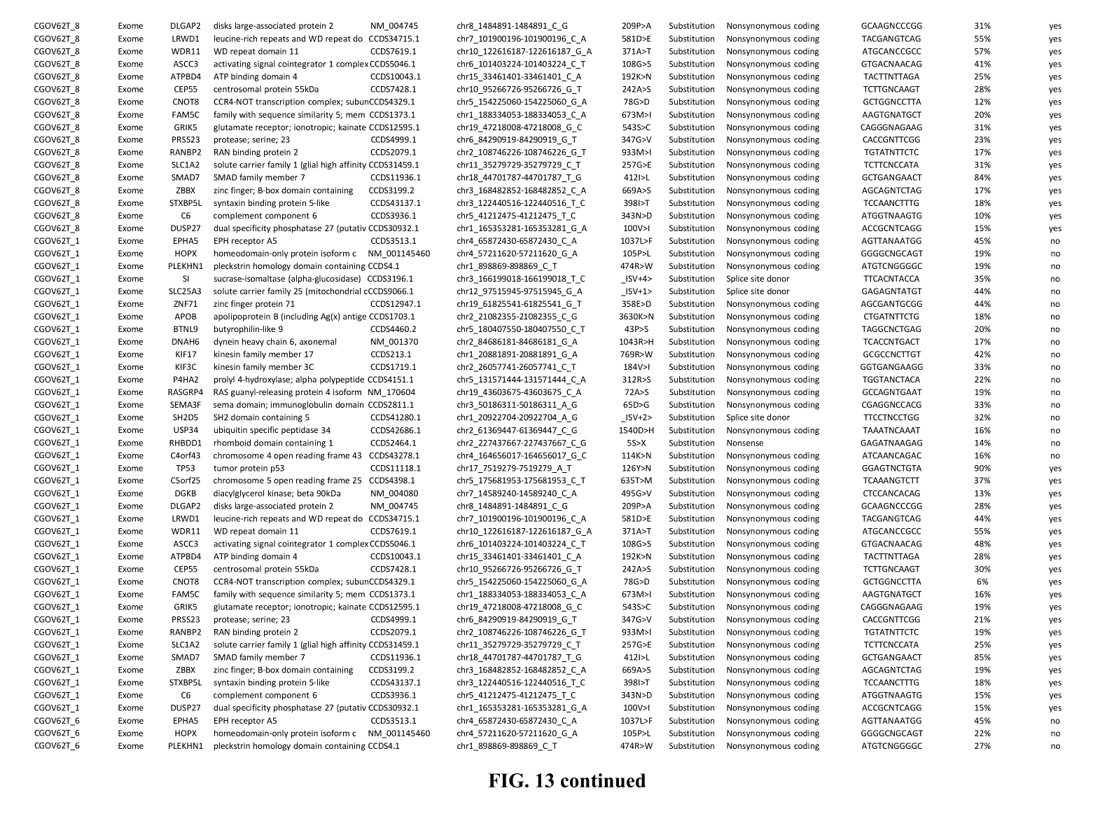

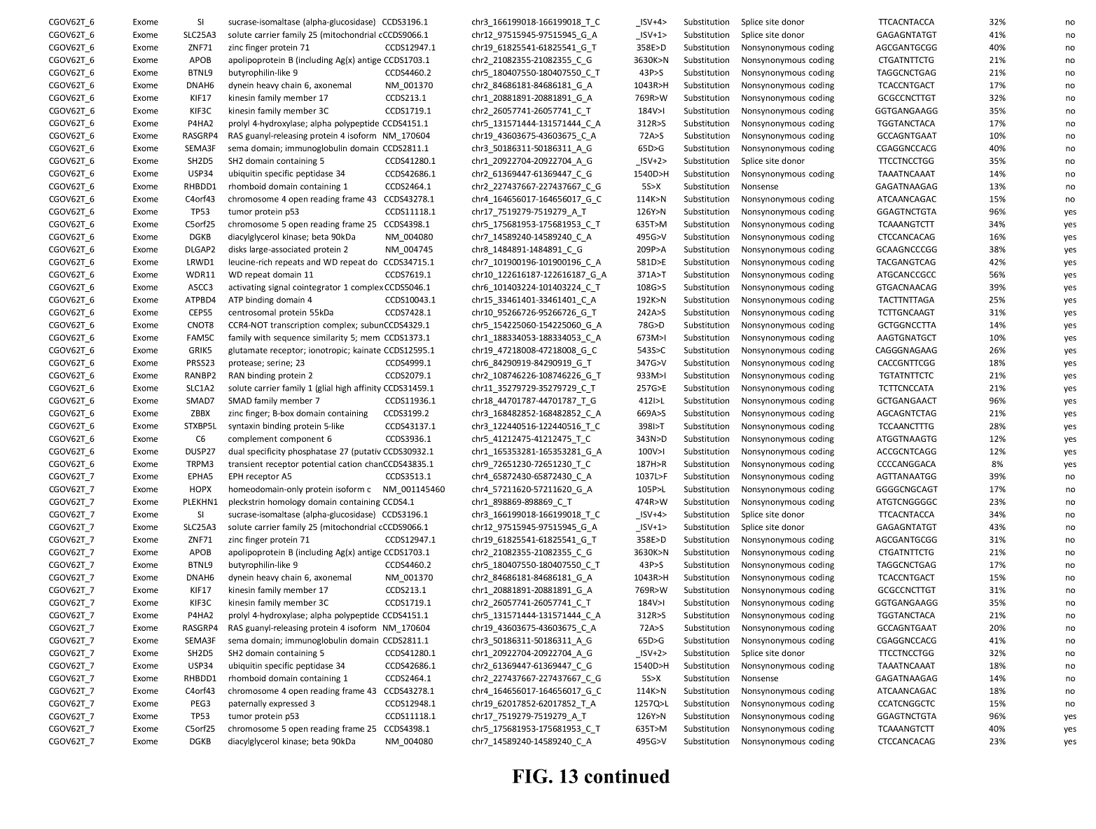

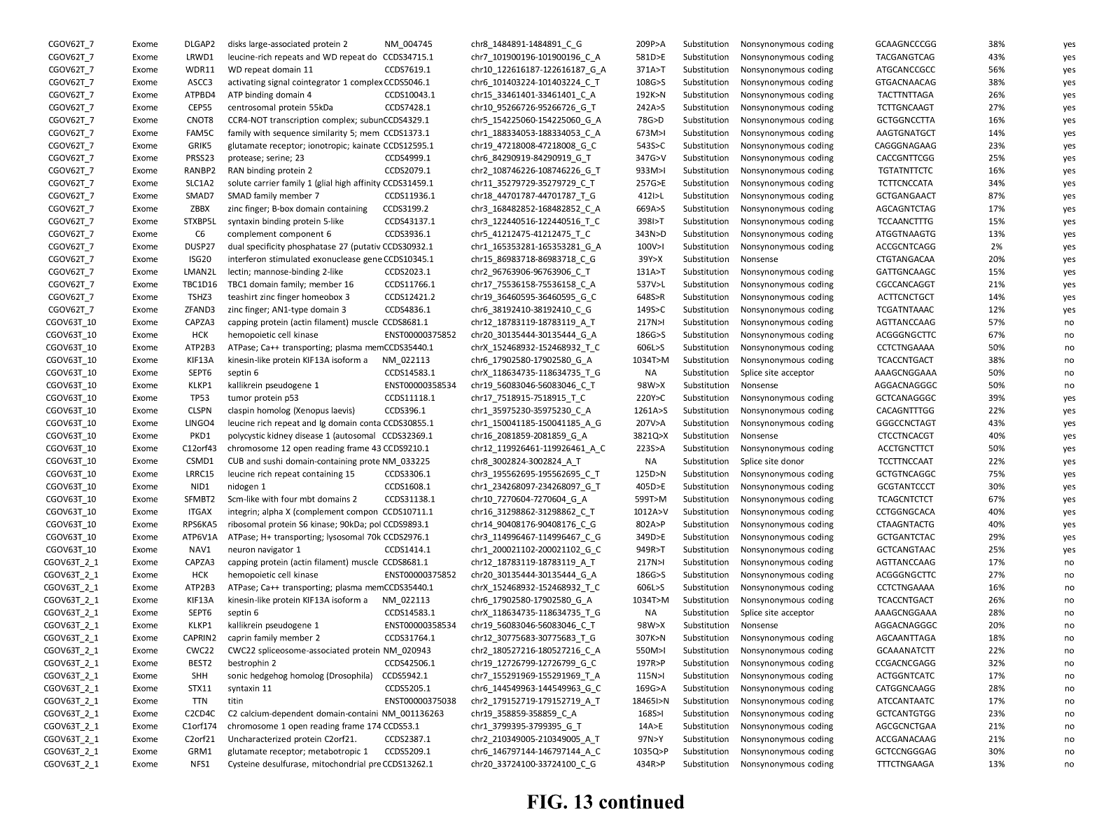

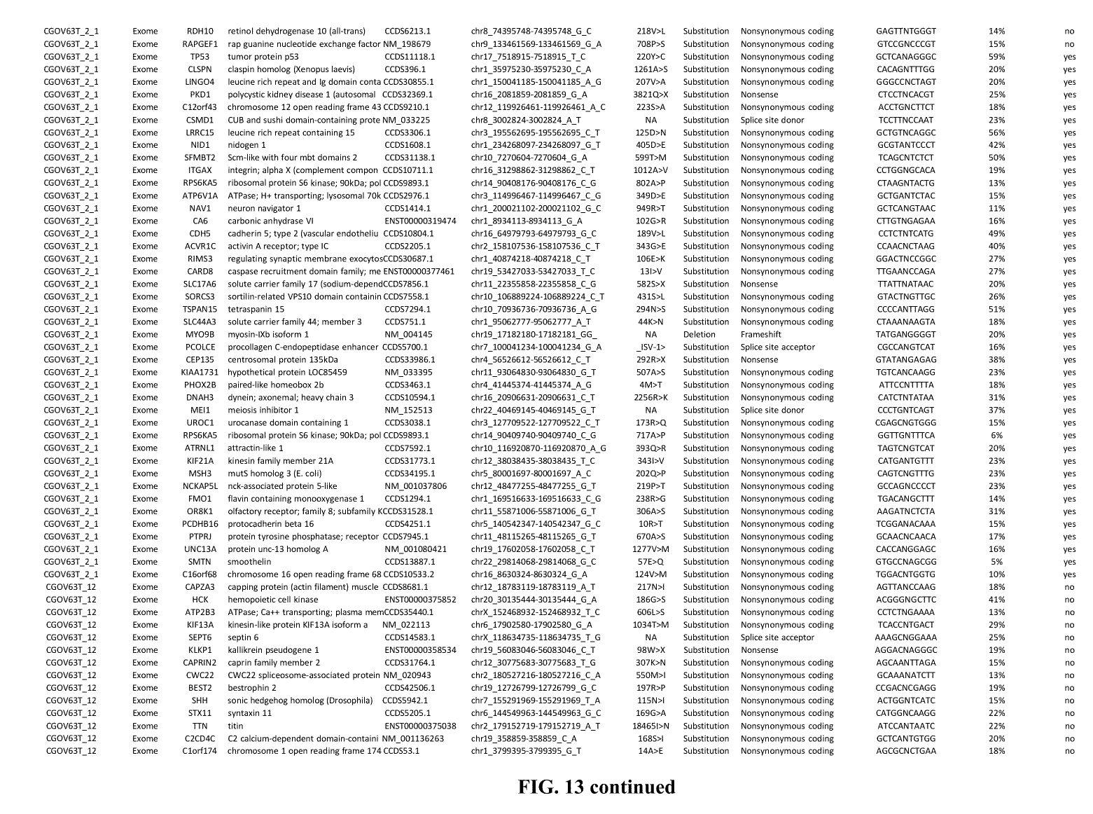

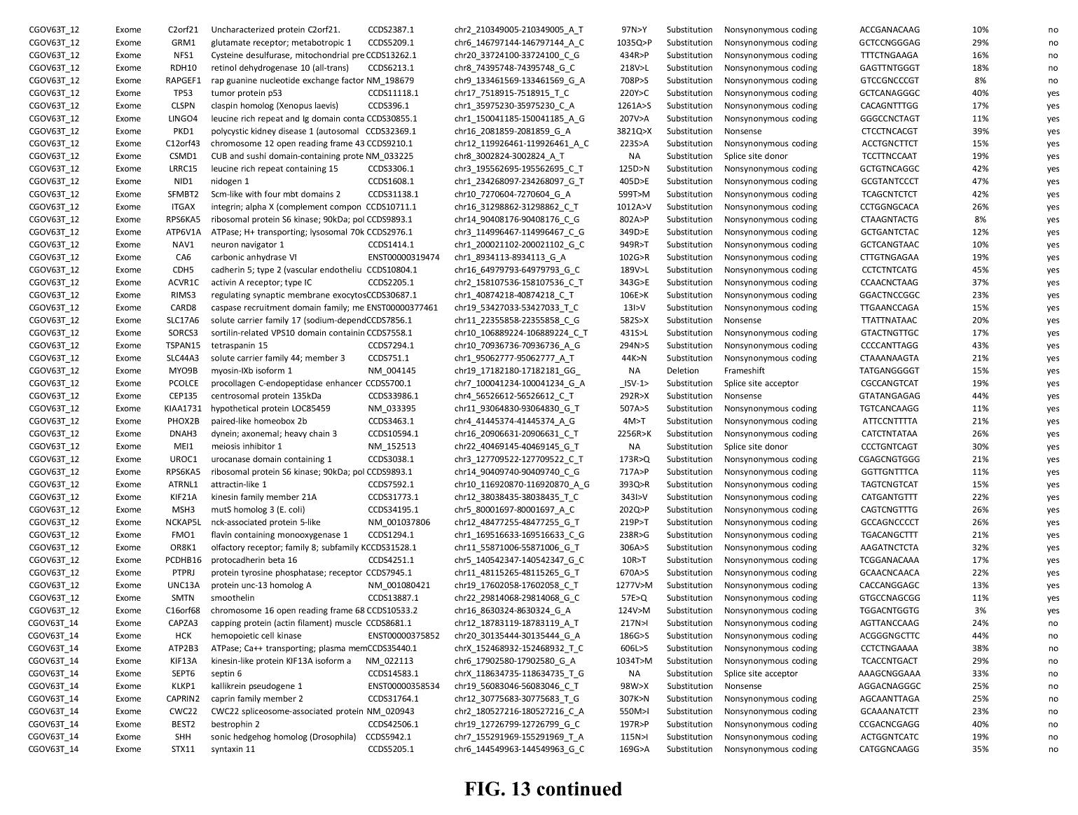

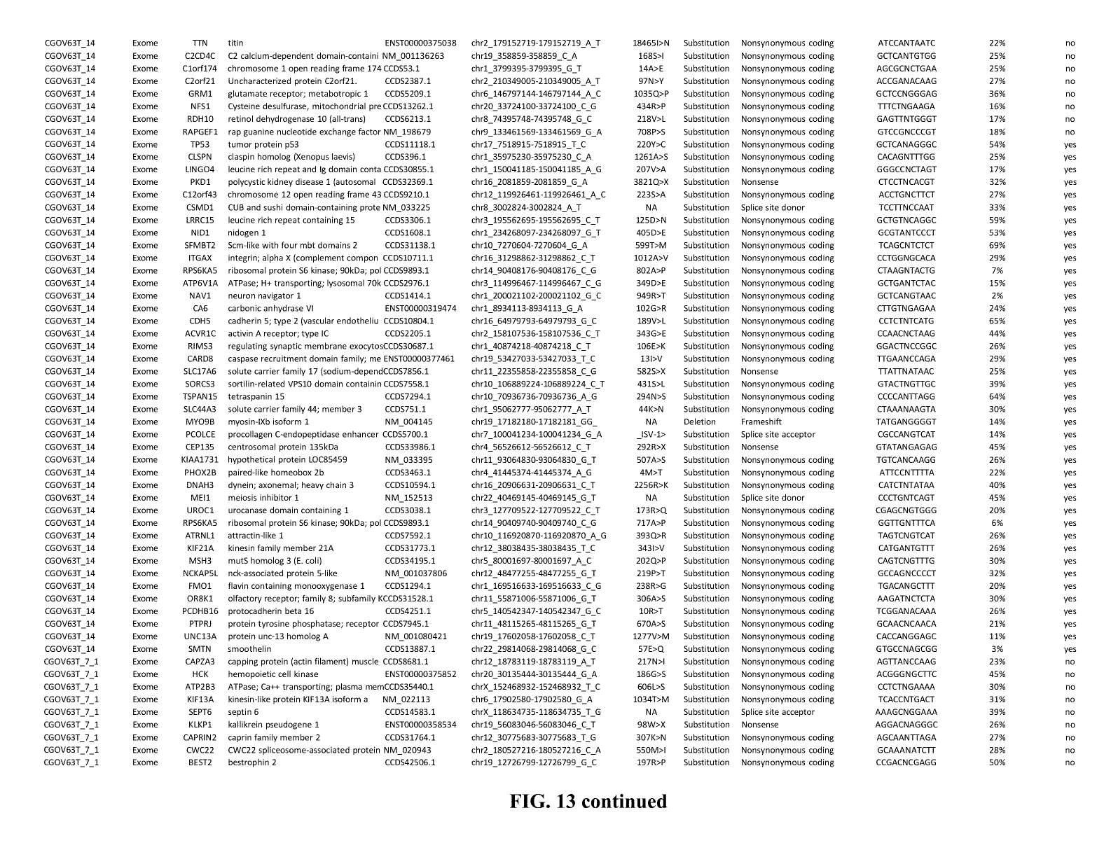

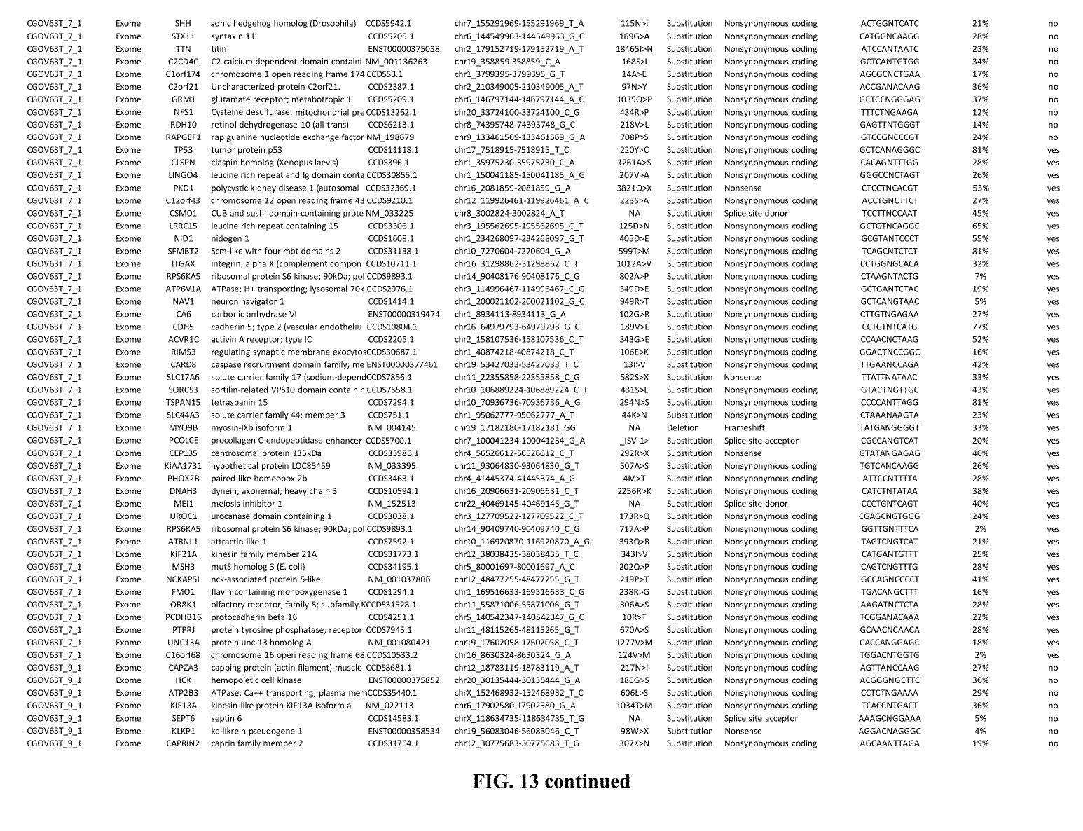

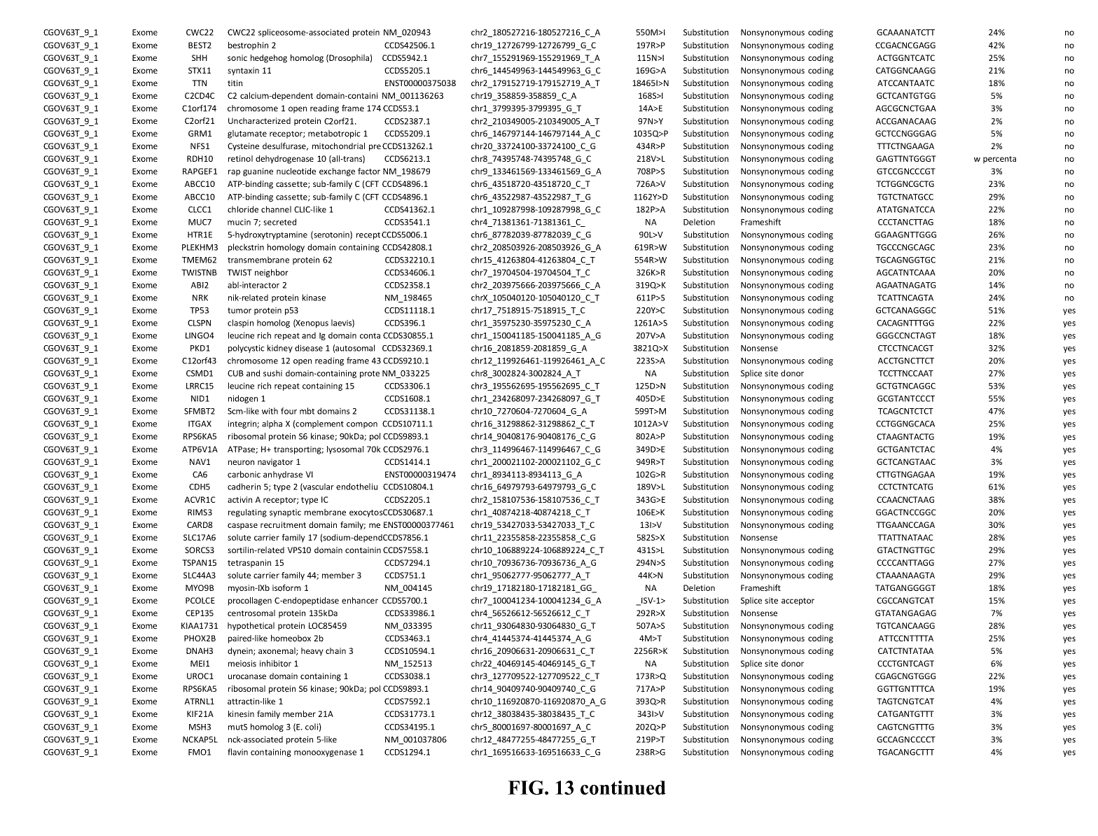

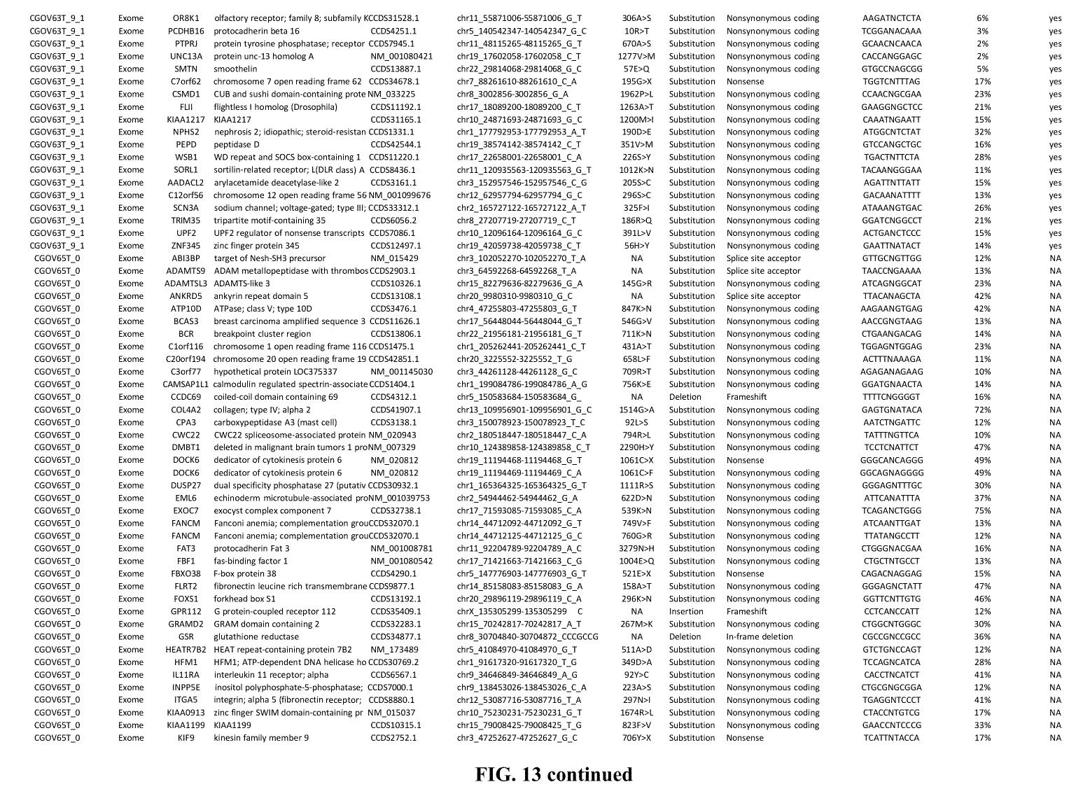

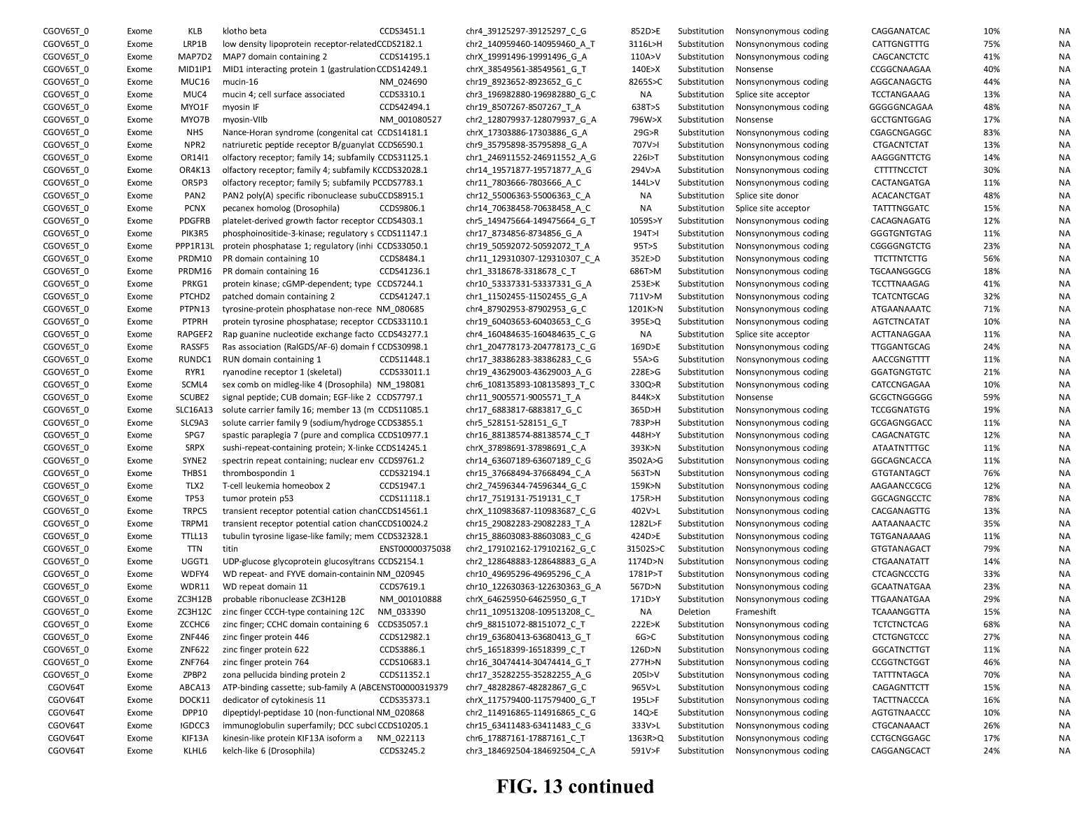

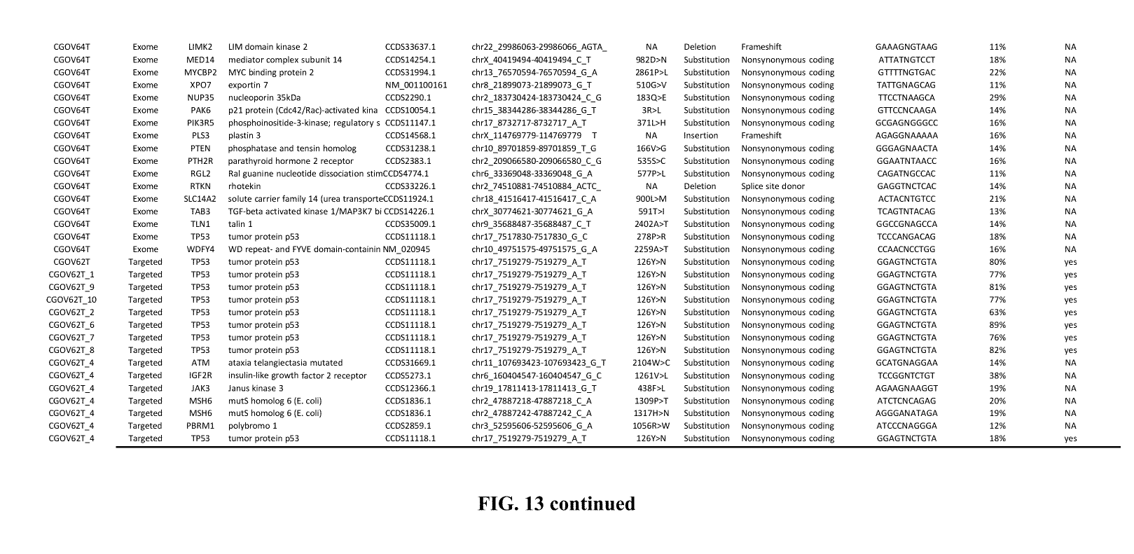

To elucidate genetic alterations in the coding regions of these cancers, we used next-generation sequencing platforms to examine the entire exomes or a set of targeted genes in matched tumor and normal specimens (FIG. 1). This approach allowed us to identify sequence changes, including single base and small insertion or deletion mutations, as well as copy number alterations in >20,000 genes in the whole-exome analyses and 120 genes in the targeted analyses (FIGS. 12-13, Supplementary Tables S2 and S3). Given the challenges of genome-wide analyses of small sample amounts, we developed experimental and bioinformatic approaches for detection of somatic alterations from laser capture microdissected tissue. These included optimized approaches for microdissection after immunohistochemical staining, improved DNA recovery from formalin fixed tissues, library construction from limited and stained tissue samples, and error correction methods in next generation sequence analyses (see Materials and Methods). We optimized these approaches using targeted methods in a subset of samples, and then used whole-exome analyses to evaluate coding sequence alterations in all samples. We obtained a total of 460 Gb of sequence data, resulting in a per-base sequence coverage of an average ˜104-fold for each tumor analyzed by whole-exome sequencing (FIG. 11).

### EXAMPLE 3

### Analysis of Sequence Changes

Using a high-sensitivity mutation detection pipeline, we identified an average of 45 sequence alterations per sample. Candidate alterations were evaluated across samples in an individual to determine if they were present in multiple neoplastic lesions or were unique to a particular sample. To allow for the possibility that a subclone may have developed in a tumor lesion prior to becoming a dominant clone at another location, we determined if genetic alterations that were present in one tumor were also present in a low fraction of neoplastic cells of other lesions. This method excluded potential artifacts related to mapping, sequencing or PCR errors, allowing specific detection of alterations present in >1% of sequence reads (See Materials and Methods for additional information).

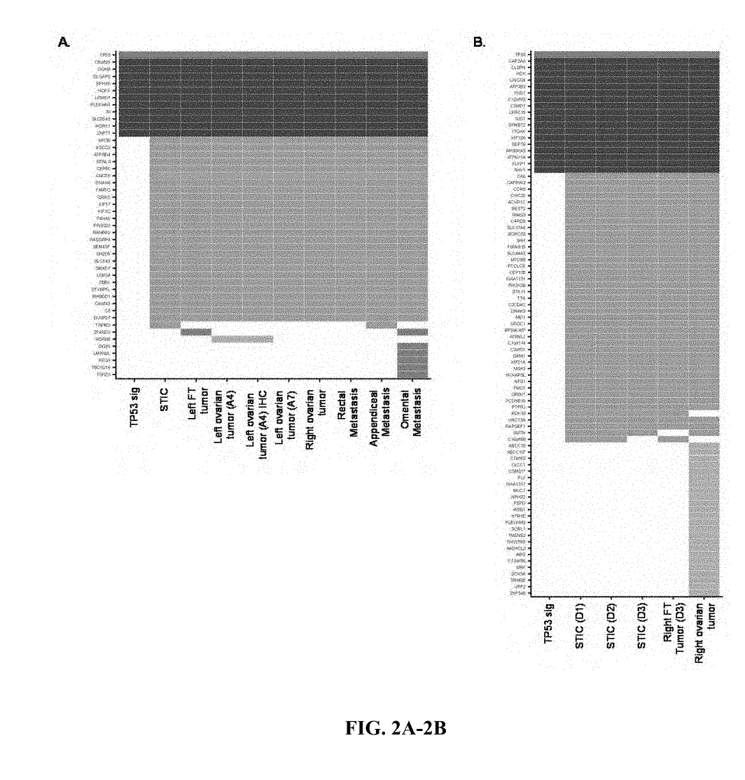

Whole-exome sequence analyses of the ten tumor samples from Patient 1 identified somatic mutations that were present in all neoplastic samples analyzed as well as specific changes that were present in individual or subsets of tumors (FIG. 2). As expected, we identified a sequence change in the TP53 tumor suppressor gene, a well-known driver gene in HGSOC. The Y126N alteration was identical in all samples analyzed including in the TP53 signature, the STIC lesion, and other tumors. These data suggest that the TP53 mutation was among the earliest initiating events for ovarian carcinoma development as all lesions harbored this alteration.

Overall, we detected a total of 46 sequence alterations among the affected lesions in Patient 1. The STIC lesions, fallopian tube tumor, left and right ovarian cancers, and three of four metastatic lesions had nearly identical changes, harboring a common set of 38 somatic mutations (FIG. 2). These results suggest that a progenitor tumor clone containing these alterations led to the development of the tumors. A few additional mutations were present in a subset of tumors, such as a change in the TRPM3 cation channel gene that was identical in the STIC and appendiceal met, and a change in the ZFAND3 zinc finger gene that was present in the fallopian tube tumor and omental met. These alterations suggest that additional changes may have occurred in the evolution of these related tumors. Interestingly, most metastatic lesions did not contain new somatic mutations compared to the STIC lesion and fallopian and ovarian tumors.

### EXAMPLE 4

### Chromosomal Alterations in Tumor Samples

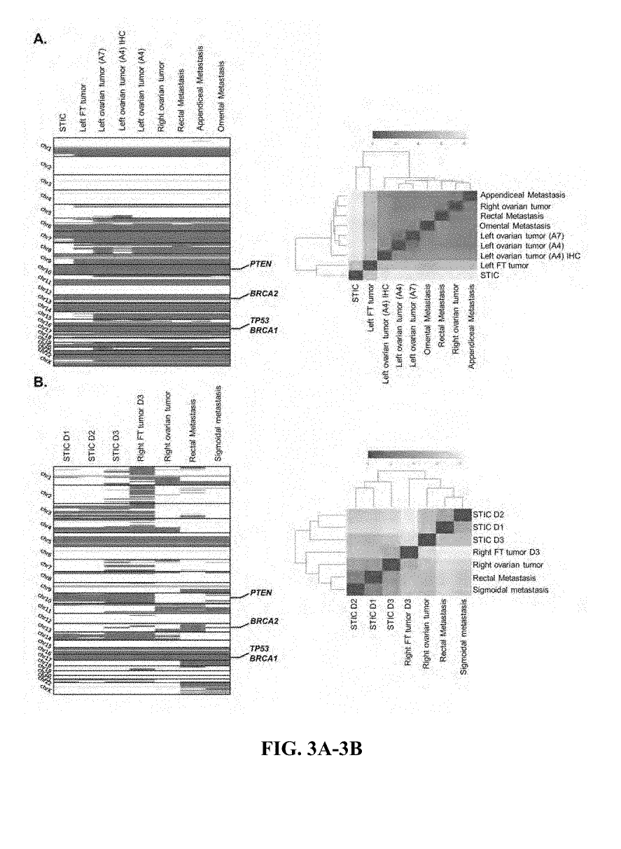

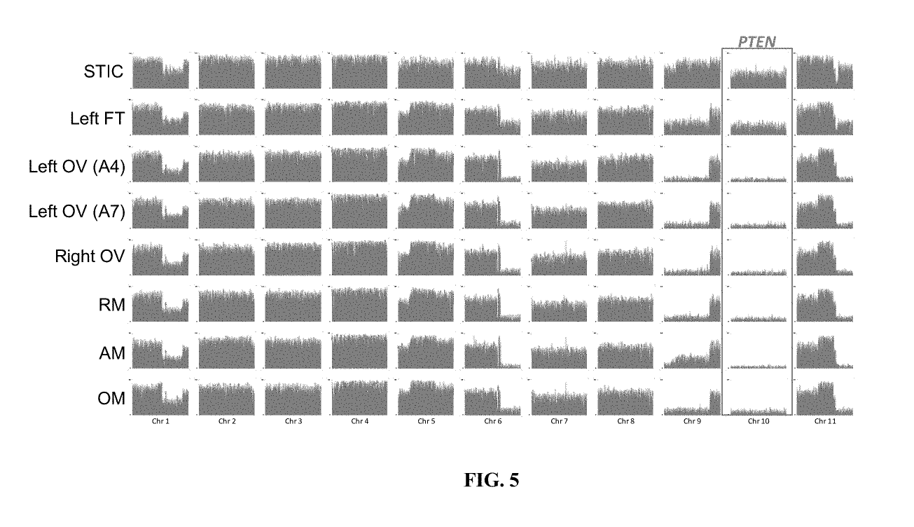

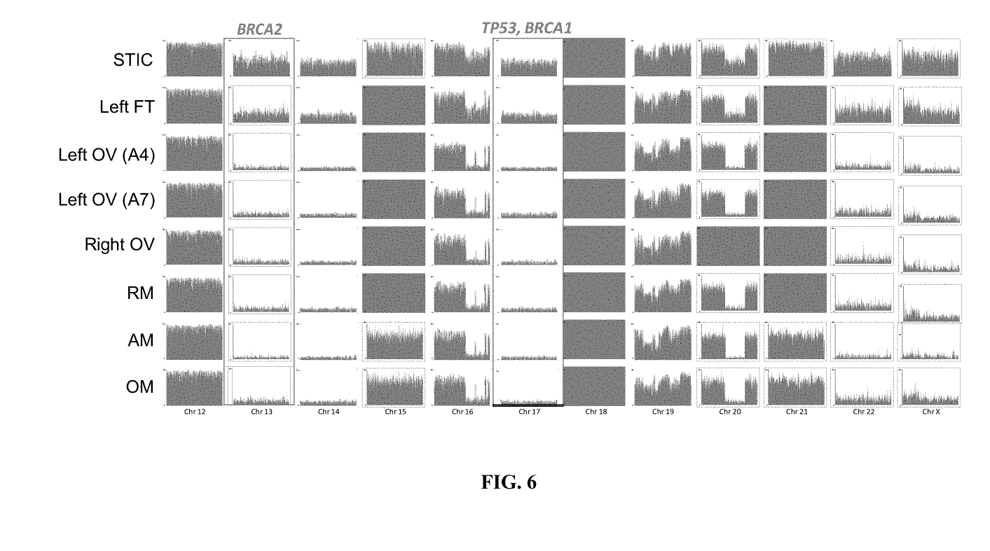

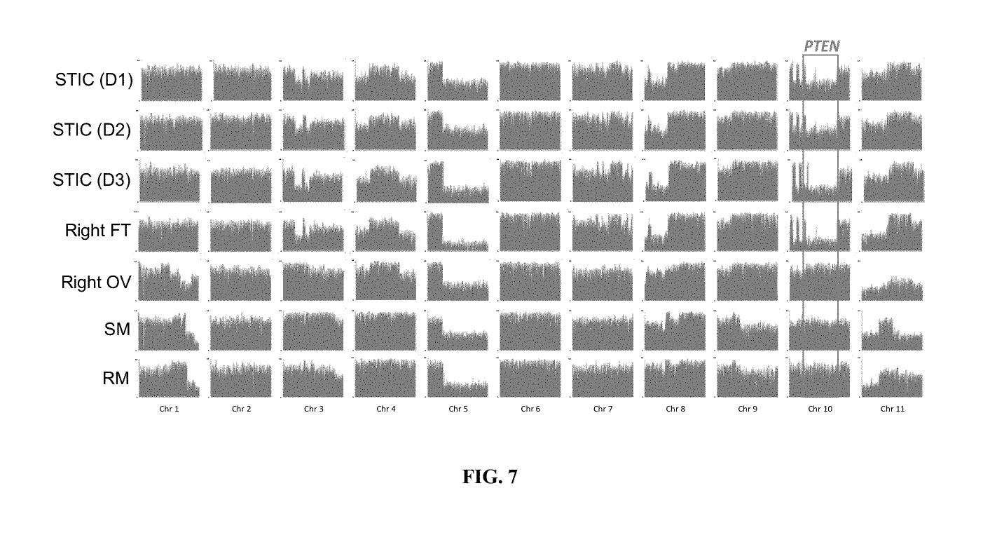

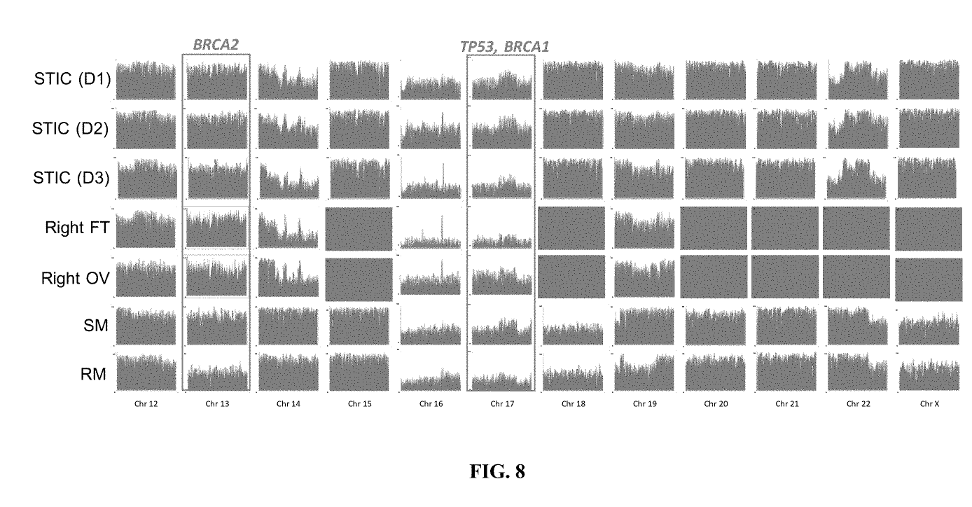

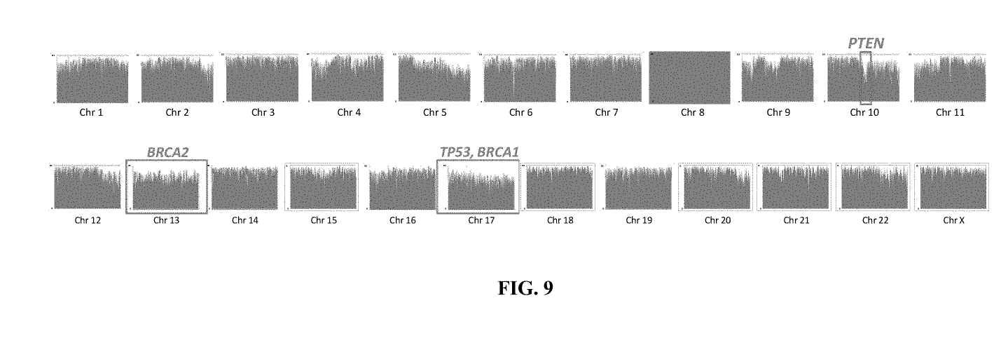

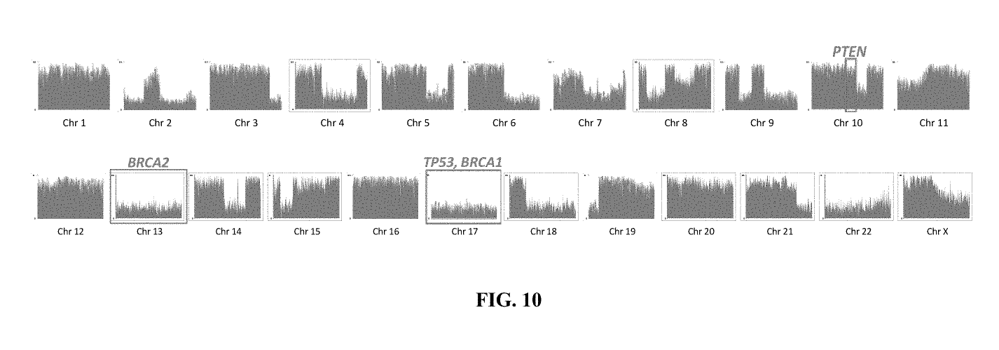

Little is known about the dynamics of chromosomal instability that drive genomic aberrations in HGSOC¹⁰. To examine chromosomal structural variation in the multiple tumors of Patient 1, we focused on regions of allelic imbalance that can result from the complete loss of an allele or from an increase in copy number of one allele relative to the other. We divided the genome into chromosome bands and for each band compared the minor allele (B-allele) frequency values in tumor and normal samples using the ˜15,000 whole-exome germline heterozygous single nucleotide polymorphisms (SNPs) observed (FIG. 3 and FIGS. 5-10). We performed this analysis in all specimens except the TP53 signature lesion where the sequence coverage was limited. Overall, we observed that ˜52% of the genome had chromosomal imbalances in the samples analyzed of Patient 1 (FIG. 3, left panel).

To investigate the relationship of cytogenetic alterations among tumor samples examined, we performed a hierarchical cluster analysis of chromosomal imbalances. Cluster dendrograms were generated using the Euclidean distance of the allelic imbalances described above (FIG. 3, right panels). Tumors with the highest degree of similarity were grouped in short-branched clusters and further connected to similar cases generating higher-order clusters of decreasing similarity as indicated by longer branches. The analysis revealed a grouping of STIC and fallopian tube tumors in one cluster and a second cluster characterized by ovarian tumors and metastatic lesions. Although all tumors had a high degree of similarity of chromosomal changes, tumors in the second cluster had acquired additional chromosomal alterations (FIG. 3A, left panel).

### EXAMPLE 5

### Evolutionary Relationship of Neoplastic Lesions

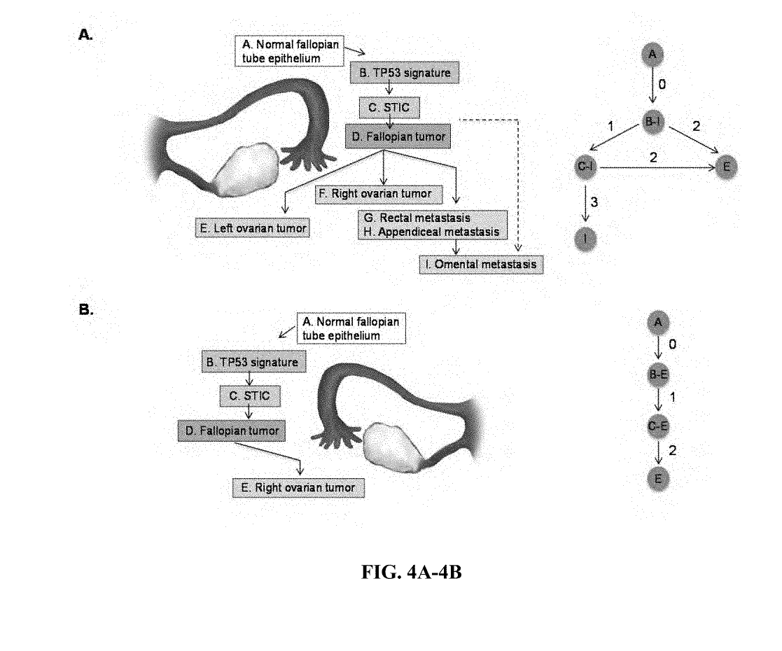

As somatic genetic alterations can be used to recreate the evolutionary history of tumor clones, we used the somatic point mutations and chromosomal aberrations observed in Patient 1 to determine the history of tumor clonal evolution in this patient. We employed a subclone hierarchy inference tool called SCHISM (SubClonal Hierarchy Inference from Somatic Mutations) which enables improved phylogenetic reconstruction by incorporating estimates of the fraction of neoplastic cells in which a mutation occurs (mutation cellularity) 23. Because genomic regions with allelic imbalances may influence the estimate of mutation cellularities, we investigated the dynamics of somatic evolution by examining alterations that were not present in regions of allelic imbalance in any sample (FIG. 4). Of the 46 somatic alterations initially detected in all lesions analyzed, 26 were present in regions of chromosomal alterations and were removed, resulting in 20 mutations that were used to construct the phylogenetic tree illustrated in FIG. 4.

A SCHISM tree node represents cells harboring a unique compartment of mutations defining a subclone whereas an edge represents a set of mutations acquired by the cells in the progeny nodes that distinguish them from the cells in the parental node. By definition, for an individual cancer there could only be one parental clone, although there could be many different progeny subclones representing invasive or metastatic lesions or further evolution of the primary tumor. The optimal hierarchy among subclones is determined by evaluating all possible relationships and identifying the simplest model that can explain the observed nodes. The mutation data suggested that the TP53 signature or STIC lesions contained the ancestral clone for the observed tumors. The STIC lesions were the likely precursors of the fallopian tube tumors (node 1), which in turn lead to left ovarian tumors (node 2), right ovarian tumors and metastatic lesions (nodes 1 and 3).

The ancestral nature of the STIC lesions was strengthened by the observation that these lesions could also serve as a direct parental clone for tumors outside the fallopian tube, including the appendiceal metastasis that shared a unique sequence change present only in in this metastasis and the STIC lesion (FIG. 2). Likewise, the fallopian tube tumor is likely a direct parental clone for the omental metastases as demonstrated by the shared alteration in ZFAND3. Overall, the phylogenetic model generated by these data suggest that the ancestral clone for the ovarian cancer in this patient developed in the fallopian tubes and had the capacity to spread both locally and to other organs while bypassing the ovaries (FIG. 4A).

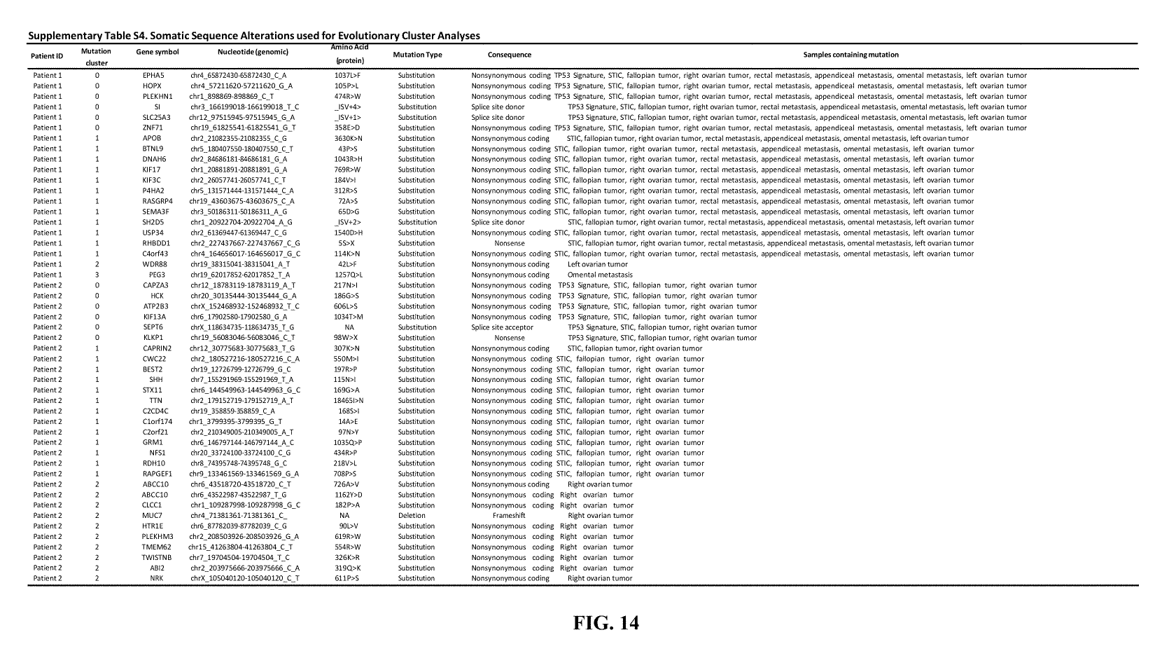

A similar evolutionary pattern was observed for Patient 2. Overall, we detected a total of 85 sequence alterations among the affected lesions. Three STIC lesions, the fallopian tube tumor and the ovarian cancers contained nearly identical sequence changes, harboring a common set of 59 somatic mutations (FIG. 2). The TP53 Y220C alteration was identical in all samples analyzed including in the TP53 signature, the STIC lesions, and other tumors. A few additional mutations were present in a subset of tumors, such as a mutation in SMTN smoothelin gene that was identical in all the STICs and the ovarian tumor, and a change in C16orf68 that was present in STIC D1, STIC D2 and the fallopian tumor. The ovarian tumor contained 24 additional somatic mutations compared to the STIC lesion and fallopian tumor. Although two metastatic lesions could not be analyzed for sequence changes due to low tumor purity, all seven tumor samples of Patient 2 were analyzed for copy number changes. Cluster dendrograms revealed a grouping of the three STICs in one cluster and a second cluster characterized by the fallopian tube tumors, the ovarian tumor and metastatic lesions (FIG. 3B, right panel, FIG. 14, Supplementary Table S4). Similar to Patient 1, the SCHISM analysis revealed that the parental clone in this patient was present in the fallopian tube and gave rise to the ovarian cancer.

To extend these analyses to patients with familial ovarian cancer, we examined neoplastic samples from two individuals with germline BRCA alterations where STIC lesions were incidentally identified after prophylactic bilateral salpingo-oophorectomy. We identified BRCA1 or BRCA2 sequence alterations in the germline of these patients (BRCA1 Q1200X and BRCA2 L2653P in Patients 3 and 4, respectively), as well as somatic mutations in TP53, and LOH of both chromosome 13 and 17, encompassing the BRCA1/BRCA2 and TP53 loci. Whole exome analyses showed that the STIC lesions contained a total of 91 and 23 somatic mutations, in Patients 3 and 4 respectively. Overall, these analyses revealed that patients with germline BRCA changes have a roughly similar number of sequence changes to patients with sporadic tumors.

### EXAMPLE 6

### Recurrent Molecular Alterations

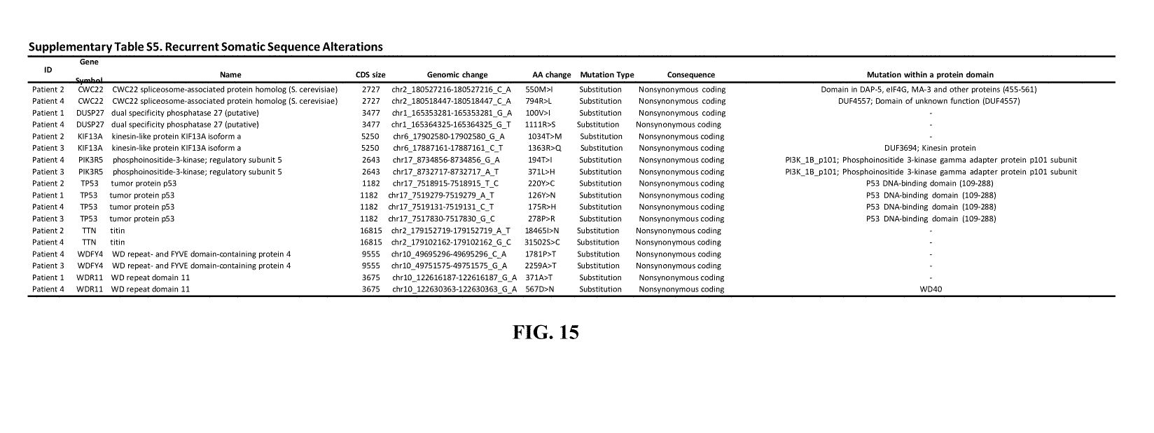

We examined tumors from the four patients to identify recurrent sequence or chromosomal changes. Although no genes other than TP53 were mutated in all patients analyzed, we identified mutations in seven genes that were altered in two or more patients (FIG. 15, Supplementary Table S5). These included mutations in the tumors of two patients of the PIK3R5 gene that encodes a regulatory subunit of the PI3-kinase complex. Patient 3 also had a somatic alteration in PTEN that together with changes in PIK3R5 highlight the importance of the PI3K pathway in ovarian cancer10.

In addition to recurrent sequence changes, we found alterations in regions of allelic imbalances encompassing several tumor suppressor genes involved in ovarian cancer. Remarkably, these included loss of BRCA1/2 and TP53 in all four patients, and loss of PTEN for Patients 1, 2 and 3 (in addition the somatic sequence alterations of these genes). In all cases, the LOH observed in the metastatic lesions and ovarian tumor lesions for regions encompassing these genes were already present in the fallopian tube tumor and STIC lesions. Considering the evolutionary model above, these data suggest that a combination of sequence changes in a few genes including TP53 together with loss of the TP53 wild-type allele as well as BRCA1/2, and PTEN may be crucial early events that are needed for the initiation of STICs24 25.

### EXAMPLE 7

### Evolutionary Timeline of Ovarian Cancer Development

To estimate the time between the development of the earliest neoplastic clones in the fallopian tube and the development of ovarian and other metastatic lesions we used a mathematical model for comparative lesion analysis22,26. This model estimates the time interval between a founder cell of a tumor of interest and the ancestral precursor cell assuming that mutation rates and cell division times are constant throughout a patient's life [see Materials and Methods]. In the case of Patient 1, this model would suggest ˜1.3 years between the development of the STIC lesion and either the fallopian tube or the ovarian tumors (90% CI, 0.2-3.8 years). Other lesions in this patient may have required even less time, as there are no mutational differences between the STIC lesion and the appendiceal metastasis (0.6 years; CI, 0 to 2.6 years), while other lesions such as the omental metastasis appear to have taken longer to develop (4.9 years; CI, 2.5 to 8.5 years). For Patient 2, although the time between development of the STIC and the fallopian tube tumor was estimated to be relatively rapid (0.4 years, CI, 0 to 2.0), a relatively long period of time was required for the development of the ovarian tumor (12.0 years, CI, 8.8 to 15.8 years).

### REFERENCES

The disclosure of each reference cited is expressly incorporated herein.

1. Ferlay J, Steliarova-Foucher E, Lortet-Tieulent J, et al. Cancer incidence and mortality patterns in Europe: estimates for 40 countries in 2012. European journal of cancer 2013; 49:1374-403.

2. Siegel R L, Miller K D, Jemal A. Cancer statistics, 2015. CA: a cancer journal for clinicians 2015; 65:5-29.

3. Dinkelspiel H E, Champer M, Hou J, et al. Long-term mortality among women with epithelial ovarian cancer. Gynecologic oncology 2015; 138:421-8.

4. Menon U, Griffin M, Gentry-Maharaj A. Ovarian cancer screening—current status, future directions. Gynecologic oncology 2014; 132:490-5.

5. Jacobs U, Menon U, Ryan A, et al. Ovarian cancer screening and mortality in the UK Collaborative Trial of Ovarian Cancer Screening (UKCTOCS): a randomised controlled trial. Lancet 2015.

6. Kurman R J, Shih Ie M. The origin and pathogenesis of epithelial ovarian cancer: a proposed unifying theory. The American journal of surgical pathology 2010; 34:433-43.

7. Karst A M, Drapkin R. Ovarian cancer pathogenesis: a model in evolution. Journal of oncology 2010; 2010:932371.

8. Levanon K, Crum C, Drapkin R. New insights into the pathogenesis of serous ovarian cancer and its clinical impact. Journal of clinical oncology: official journal of the American Society of Clinical Oncology 2008; 26:5284-93.

9. Bowtell D D, Bohm S, Ahmed A A, et al. Rethinking ovarian cancer II: reducing mortality from high-grade serous ovarian cancer. Nature reviews Cancer 2015; 15:668-79.

10. Cancer Genome Atlas Research N. Integrated genomic analyses of ovarian carcinoma. Nature 2011; 474:609-15.

11. Patch A M, Christie E L, Etemadmoghadam D, et al. Whole-genome characterization of chemoresistant ovarian cancer. Nature 2015; 521:489-94.

12. Cass I, Holschneider C, Datta N, Barbuto D, Walts AE, Karlan BY. BRCA-mutation-associated fallopian tube carcinoma: a distinct clinical phenotype? Obstetrics and gynecology 2005; 106:1327-34.

13. Piek J M, Verheij en R H, Kenemans P, Massuger L F, Bulten H, van Diest P J. BRCA1/2-related ovarian cancers are of tubal origin: a hypothesis. Gynecologic oncology 2003; 90:491.

14. Piek J M, van Diest P J, Zweemer R P, et al. Dysplastic changes in prophylactically removed Fallopian tubes of women predisposed to developing ovarian cancer. The Journal of pathology 2001; 195:451-6.

15. Medeiros F, Muto M G, Lee Y, et al. The tubal fimbria is a preferred site for early adenocarcinoma in women with familial ovarian cancer syndrome. The American journal of surgical pathology 2006; 30:230-6.

16. Lee Y, Miron A, Drapkin R, et al. A candidate precursor to serous carcinoma that originates in the distal fallopian tube. The Journal of pathology 2007; 211:26-35.

17. Kindelberger D W, Lee Y, Miron A, et al. Intraepithelial carcinoma of the fimbria and pelvic serous carcinoma: Evidence for a causal relationship. The American journal of surgical pathology 2007; 31:161-9.

18. Kuhn E, Kurman R J, Vang R, et al. TP53 mutations in serous tubal intraepithelial carcinoma and concurrent pelvic high-grade serous carcinoma--evidence supporting the clonal relationship of the two lesions. The Journal of pathology 2012; 226:421-6.

19. McDaniel A S, Stall J N, Hovelson D H, et al. Next-Generation Sequencing of Tubal Intraepithelial Carcinomas. JAMA oncology 2015; 1:1128-32.

20. Bashashati A, Ha G, Tone A, et al. Distinct evolutionary trajectories of primary high-grade serous ovarian cancers revealed through spatial mutational profiling. The Journal of pathology 2013; 231:21-34.

21. Nik N N, Vang R, Shih Ie M, Kurman R J. Origin and pathogenesis of pelvic (ovarian, tubal, and primary peritoneal) serous carcinoma. Annual review of pathology 2014; 9:27-45.

22. Yachida S, Jones S, Bozic I, et al. Distant metastasis occurs late during the genetic evolution of pancreatic cancer. Nature 2010; 467:1114-7.

23. Niknafs N, Beleva-Guthrie V, Naiman D Q, Karchin R. SubClonal Hierarchy Inference from Somatic Mutations: Automatic Reconstruction of Cancer Evolutionary Trees from Multi-region Next Generation Sequencing. PLoS computational biology 2015; 11:e1004416.

24. Roh M H, Yassin Y, Miron A, et al. High-grade fimbrial-ovarian carcinomas are unified by altered p53, PTEN and PAX2 expression. Modern pathology: an official journal of the United States and Canadian Academy of Pathology, Inc 2010; 23:1316-24.

25. Perets R, Wyant G A, Muto K W, et al. Transformation of the fallopian tube secretory epithelium leads to high-grade serous ovarian cancer in Brca; Tp53;Pten models. Cancer cell 2013; 24:751-65.

26. Jones S, Chen W D, Parmigiani G, et al. Comparative lesion sequencing provides insights into tumor evolution. Proceedings of the National Academy of Sciences of the United States of America 2008; 105:4283-8.

27. Perets R, Drapkin R. It's Totally Tubular . . . Riding The New Wave of Ovarian Cancer Research. Cancer research 2016; 76:10-7.

28. Conner J R, Meserve E, Pizer E, et al. Outcome of unexpected adnexal neoplasia discovered during risk reduction salpingo-oophorectomy in women with germ-line BRCA1 or BRCA2 mutations. Gynecol Oncol 2014; 132:280-6.

29. Rebbeck T R, Lynch H T, Neuhausen S L, et al. Prophylactic oophorectomy in carriers of BRCA1 or BRCA2 mutations. The New England journal of medicine 2002; 346:1616-22.

30. Kauff N D, Satagopan J M, Robson M E, et al. Risk-reducing salpingo-oophorectomy in women with a BRCA1 or BRCA2 mutation. The New England journal of medicine 2002; 346:1609-15.

31. Falconer H, Yin L, Gronberg H, Altman D. Ovarian cancer risk after salpingectomy: a nationwide population-based study. J Natl Cancer Inst 2015; 107.

32. McAlpine J N, Hanley G E, Woo M M, et al. Opportunistic salpingectomy: uptake, risks, and complications of a regional initiative for ovarian cancer prevention. American journal of obstetrics and gynecology 2014; 210:471 el-11.

33. Kwon J S, Tinker A, Pansegrau G, et al. Prophylactic salpingectomy and delayed oophorectomy as an alternative for BRCA mutation carriers. Obstetrics and gynecology 2013; 121:14-24.

34. Parker W H, Broder M S, Chang E, et al. Ovarian conservation at the time of hysterectomy and long-term health outcomes in the nurses' health study. Obstetrics and gynecology 2009; 113:1027-37.

35. Longacre T A, Oliva E, Soslow R A, Association of Directors of A, Surgical P. Recommendations for the reporting of fallopian tube neoplasms. Hum Pathol 2007; 38:1160-3.

36. Haber D A, Velculescu V E. Blood-based analyses of cancer: circulating tumor cells and circulating tumor DNA. Cancer discovery 2014; 4:650-61.

37. Kinde I, Bettegowda C, Wang Y, et al. Evaluation of DNA from the Papanicolaou test to detect ovarian and endometrial cancers. Science translational medicine 2013; 5:167ra4.

38. Eberle F C, Hanson J C, Killian J K, et al. Immunoguided laser assisted microdissection techniques for DNA methylation analysis of archival tissue specimens. The Journal of molecular diagnostics: JMD 2010; 12:394-401.

39. Bertotti A, Papp E, Jones S, et al. The genomic landscape of response to EGFR blockade in colorectal cancer. Nature 2015; 526:263-7.

40. Jones S, Anagnostou V, Lytle K, et al. Personalized genomic analyses for cancer mutation discovery and interpretation. Science translational medicine 2015; 7:283ra53.

We claim:

1. A method for reducing risk of ovarian cancer, comprising:
offering or recommending to a patient at high risk of ovarian cancer an option for surgical removal of the fallopian tubes without oophorectomy; wherein the patient is classified as high risk when the patient presents with one or more of the following factors selected from the group consisting of: a) the patient has an inherited defect in BRCA1 and/or BRCA2; b) advanced age; c) obesity; d) reproductive history; e) fertility drug use; f) androgen use; g) estrogen use; h)family cancer syndrome; i) HNPCC mutation in one or more of MLH1, MLH3, MSH2, MSH6, TGFBR2, PMS1, or PMS2; j) Putz-Jeghers syndrome; k) MUTYH-associated polyposis; and l) personal history of breast cancer.

2. A method for reducing risk of ovarian cancer, comprising:
offering or recommending to a patient who is a candidate for obtaining a tubal ligation as a contraceptive measure, an option for surgical removal of the fallopian tubes without oophorectomy.

3. A method for reducing risk of omental cancer or metastasis, comprising:
offering or recommending to a patient who is a candidate for obtaining a hysterectomy for a benign cause, an option for surgical removal of the fallopian tubes without oophorectomy.

4. A method for detecting an increased risk of ovarian cancer and metastases, comprising:
conducting an examination of at least 3 sections of a pair of removed fallopian tubes, wherein removal is for a benign condition, a risk-reducing bilateral salpingectomy, or a gynecological cancer.

5. A method of characterizing a lesion in fallopian tubes or ovaries of a patient, comprising:
testing for and detecting in a sample of the lesion loss of heterozygosity of a marker selected from the group consisting of p53, PTEN, BRCA1, and BRCA2.

6. The method of claim 7 wherein loss of heterozygosity of at least two of the markers is detected.

7. A method of characterizing a lesion in fallopian tube of a patient, comprising:
testing for and detecting a mutation in a gene selected from the group consisting of: CWC22, DUSP27, KIF13A, PIK3R5, TTN, WDFY4, and WDR11.

8. The method of claim 9 further comprising testing for and detecting a mutation in TP53.

9. The method of claim 9 wherein the mutation is a substitution mutation that is a non-synonymous coding mutation.

10. The method of claim 9 further comprising testing for and detecting for a mutation in a gene selected from the group consisting of those shown in FIG. 14.

11. The method of claim 9 further comprising testing for and detecting a mutation in a gene shown in FIG. 13.

12. A method of detecting or characterizing a lesion in fallopian tube of a patient, comprising:
testing for and detecting in a PAP smear or liquid PAP smear sample a mutation in a gene selected from the group consisting of: CWC22, DUSP27, KIF13A, PIK3R5, TTN, WDFY4, and WDR11.
